(19) United States
(12) Patent Application Publication
(10) Pub. No.: US 2025/0257054 A1
(43) Pub. Date: Aug. 14, 2025

(54) SALT FORMS OF A 4H-PYRAN-4-ONE STRUCTURED CYP11 A1 INHIBITOR

(71) Applicant: ORION CORPORATION, Espoo (FI)

(72) Inventors: David DIN BELLE, Espoo (FI); Oskari KARJALAINEN, Espoo (FI); Miika KARJOMAA, Espoo (FI); Mihaela POP, Amsterdam (NL); Petteri RUMMAKKO, Espoo (FI); Anna SHEVCHENKO, Horsholm (DK); Kai SINERVO, Espoo (FI)

(21) Appl. No.: 18/704,868

(22) PCT Filed: Oct. 27, 2022
(86) PCT No.: PCT/FI2022/050704

§ 371 Date: Apr. 25, 2024

(30) Foreign Application Priority Data
Oct. 28, 2021 (FI) 20217161

(51) Int. Cl. C07D 405/14

(52) U.S. Cl. CPC C07D 405/14

(57) ABSTRACT

The present invention relates to novel salts, particularly crystalline salts, of 5-((1-(methylsulfonyl)piperidin-4-yl)methoxy)-2-((5-(trifluoromethyl)isoindolin-2-yl)methyl)-4H-pyran-4-one (I) which are particularly suitable for use in the manufacture of pharmaceutical compositions. Furthermore, the invention relates to pharmaceutical compositions comprising such novel salts. Compound (I) is a selective inhibitor of CYP11A1 enzyme and is useful in the treatment of hormonally regulated cancers, such as prostate cancer and breast cancer.

### TECHNICAL FIELD

The present invention relates to novel salts of 5-((1-(methylsulfonyl)-piperidin-4-yl)methoxy)-2-((5-(trifluoromethyl)isoindolin-2-yl)methyl)-4H-pyran-4-one (I) and to preparation thereof. Furthermore, the invention relates to pharmaceutical compositions comprising such novel salts.

### BACKGROUND OF THE INVENTION

The compound 5-((1-(methylsulfonyl)piperidin-4-yl)methoxy)-2-((5-(tri-fluoromethyl)isoindolin-2-yl)methyl)-4H-pyran-4-one of formula (I), also referred here as compound (I), and derivatives thereof have been disclosed in WO 2018/115591. Compound (I) is a selective inhibitor of CYP11A1 enzyme and is useful in the treatment of hormonally regulated cancers, such as prostate cancer and breast cancer.

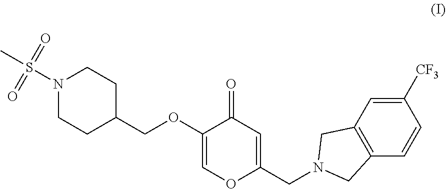 [SMILES: CS(=O)(=O)N1CCC(COc2coc(CN3Cc4ccc(C(F)(F)F)cc4C3)cc2=O)CC1]

Typically, to enable the efficient development of solid dosage forms, a form of the active ingredient is sought that has a balance of desired properties, such as crystallinity, lack of polymorphism, high melting point, solid-state stability, compressibility and lack of hygroscopicity together with satisfactory solubility. For example, it is desired that a form of the active ingredient, which has the requisite bioavailability, also has sufficient stability such that it does not degrade or convert during manufacture or storage of the pharmaceutical composition to a different form, which has different properties.

Thus, one or more forms of compound (I) are desired having properties and stability that allow a large scale manufacture of marketable pharmaceutical product suitable for the treatment of diseases such as cancer.

### SUMMARY OF THE INVENTION

It has been found that compound (I) can exist in one or more crystalline salt forms that have necessary properties, including stability and processability, that allow their use in large scale manufacture of pharmaceutical products such as tablets or capsules.

In one aspect, the present disclosure provides a salt of compound (I) with p-toluenesulfonic acid, 2-naphthalenesulfonic acid, hydrobromic acid, hydrochloric acid, methanesulfonic acid, benzenesulfonic acid, oxalic acid, phosphoric acid and maleic acid.

In another aspect, the present disclosure provides the above salts in crystalline form.

In particular, the present disclosure provides crystalline form 1 and 2 of p-toluenesulfonic acid salt, crystalline form 1 of 2-naphthalenesulfonic acid salt and crystalline form 1 of hydrobromic acid salt of compound (I). These salts are highly crystalline, exhibit particularly high melting point and excellent stability during manufacture and storage of pharmaceutical dosage forms such as, for example, wet granulated or direct compressed tablets.

In another aspect, the present disclosure provides a method for the treatment of diseases where CYP11A1 inhibition is desired, particularly in the treatment of hormonally regulated cancers, such as prostate cancer and breast cancer, comprising administering to a subject in need thereof a therapeutically effective amount of any of the above salts of compound (I) or a crystalline form thereof.

In yet another aspect, the present disclosure provides pharmaceutical compositions, particularly in the form of a tablet or a capsule, comprising any of the above salts of compound (I) or a crystalline for thereof together with one or more excipients. In yet another aspect, the present disclosure provides such compositions for use in the treatment of hormonally regulated cancers, such as prostate cancer and breast cancer.

### BRIEF DESCRIPTION OF THE DRAWINGS

FIG. 1 shows the X-ray powder diffraction (XRPD) pattern of the crystalline form 1 of compound (I).

FIG. 2 shows the differential scanning calorimetry (DSC) thermogram of the crystalline form 1 of compound (I).

FIG. 3 shows the X-ray powder diffraction (XRPD) pattern of the crystalline form 1 of p-toluenesulfonic acid salt of compound (I).

FIG. 4 shows the differential scanning calorimetry (DSC) thermogram of the crystalline form 1 of p-toluenesulfonic acid salt of compound (I).

FIG. 5 shows the X-ray powder diffraction (XRPD) pattern of the crystalline form 2 of p-toluenesulfonic acid salt of compound (I).

FIG. 6 shows the differential scanning calorimetry (DSC) thermogram of the crystalline form 2 of p-toluenesulfonic acid salt of compound (I).

FIG. 7 shows the X-ray powder diffraction (XRPD) pattern of the crystalline form 3 of p-toluenesulfonic acid salt of compound (I).

FIG. 8 shows the X-ray powder diffraction (XRPD) pattern of the crystalline form 1 of 2-naphthalenesulfonic acid salt of compound (I).

FIG. 9 shows the differential scanning calorimetry (DSC) thermogram of the crystalline form 1 of 2-naphthalenesulfonic acid salt of compound (I).

FIG. 10 shows the X-ray powder diffraction (XRPD) pattern of the crystalline form 1 of hydrobromic acid salt of compound (I).

FIG. 11 shows the differential scanning calorimetry (DSC) thermogram of the crystalline form 1 of hydrobromic acid salt of compound (I).

FIG. 12 shows the X-ray powder diffraction (XRPD) pattern of the crystalline form 1 of hydrochloric acid salt of compound (I).

FIG. 13 shows the differential scanning calorimetry (DSC) thermogram of the crystalline form 1 of hydrochloric acid salt of compound (I).

FIG. 14 shows the X-ray powder diffraction (XRPD) pattern of the crystalline form 1 of methanesulfonic acid salt of compound (I).

FIG. 15 shows the differential scanning calorimetry (DSC) thermogram of the crystalline form 1 of methanesulfonic acid salt of compound (I).

FIG. 16 shows the X-ray powder diffraction (XRPD) pattern of the crystalline form 1 of phosphoric acid salt of compound (I).

FIG. 17 shows the differential scanning calorimetry (DSC) thermogram of the crystalline form 1 of phosphoric acid salt of compound (I).

FIG. 18 shows the X-ray powder diffraction (XRPD) pattern of the crystalline form 1 of maleic acid salt of compound (I).

FIG. 19 shows the X-ray powder diffraction (XRPD) pattern of the crystalline form 1 of benzenesulfonic acid salt of compound (I).

FIG. 20 shows the differential scanning calorimetry (DSC) thermogram of the crystalline form 1 of benzenesulfonic acid salt of compound (I).

FIG. 21 shows the X-ray powder diffraction (XRPD) pattern of the crystalline form 1 of oxalic acid salt of compound (I).

FIG. 22 shows the X-ray powder diffraction (XRPD) pattern of the crystalline form 2 of oxalic acid salt of compound (I).

FIG. 23 shows the X-ray powder diffraction (XRPD) pattern of the crystalline form 3 of oxalic acid salt of compound (I).

FIG. 24 shows the X-ray powder diffraction (XRPD) pattern of the crystalline form 4 of oxalic acid salt of compound (I).

### DETAILED DESCRIPTION OF THE INVENTION

### Amorphous Compound (I)

In one aspect, the present disclosure provides an amorphous form of 5-((1-(methylsulfonyl)piperidin-4-yl)methoxy)-2-((5-(trifluoromethyl)isoindolin-2-yl)-methyl)-4H-pyran-4-one (I).

Amorphous form of compound (I) can be obtained, for example, by first preparing a concentrated solution of compound (I) in a suitable solvent such as 2-butanone. Such concentrated solution can be prepared by dissolving compound (I) in 2-butanone at room temperature under stirring to reach a concentration, for example, of about 20 mg/ml. The solvent can then be removed under vacuum, for example at room temperature, followed by recovering the amorphous solids. The amorphous form of compound (I) has been characterized by X-ray powder diffraction (XRPD) studies (data not shown).

### Crystalline Compound (I)

In one aspect, the present disclosure provides crystalline form 1 of 5-((1-(methylsulfonyl)piperidin-4-yl)methoxy)-2-((5-(trifluoromethyl)isoindolin-2-yl)-methyl)-4H-pyran-4-one (I).

Compound (I) appears to exist in a single crystalline form, here named as crystalline form 1. No other crystalline forms have been found for compound (I).

Crystalline form 1 of compound (I) can be prepared, for example, by dissolving compound (I) in a suitable solvent such as 1-propanol under heating, for example at about 50° C., until complete dissolution followed by cooling the mixture and ageing at lower temperature, for example at about 0-5° C. for about 24 hours. The mixture is dried, for example, under vacuum at about 40° C., to obtain the crystalline form 1.

The crystalline form 1 of compound (I) has been characterized by X-ray powder diffraction (XRPD) studies.

Accordingly, in one aspect, the present disclosure provides crystalline form 1 of compound (I) having a X-ray powder diffraction pattern comprising characteristic peaks at about 8.3, 12.4, 14.6, 15.5, 17.1 and 20.6 degrees 2-theta.

In yet another aspect, the present disclosure provides crystalline form 1 of compound (I) having a X-ray powder diffraction pattern comprising characteristic peaks at about 8.3, 12.4, 14.6, 15.5, 16.5, 17.1, 17.3, 20.6, 21.9, and 25.8 degrees 2-theta.

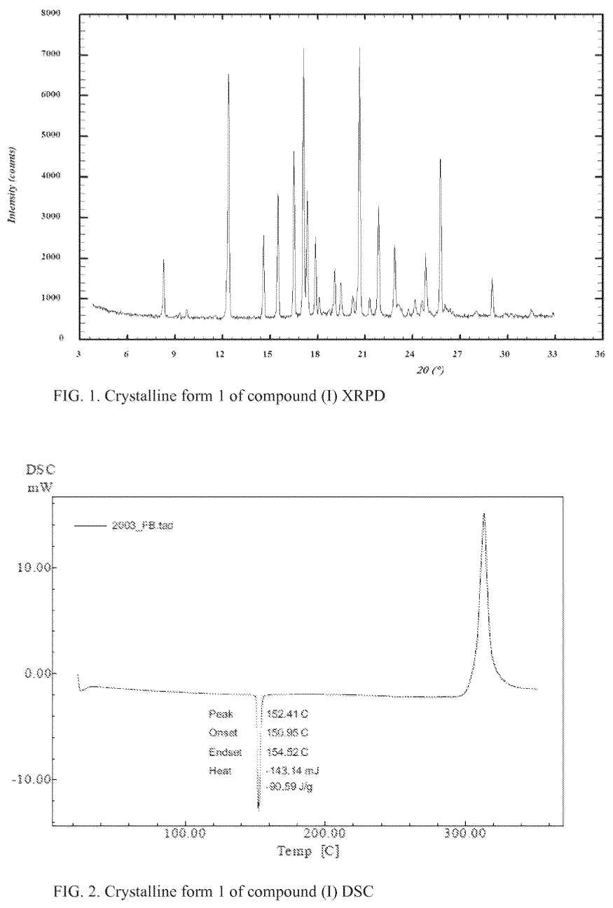

In a further aspect, the crystalline form 1 of compound (I) is further characterized by an X-ray powder diffraction pattern as depicted in FIG. 1.

Salt with p-Toluenesulfonic Acid

In one aspect, the present disclosure provides a salt of 5-((1-(methylsulfonyl)-piperidin-4-yl)methoxy)-2-((5-(trifluoromethyl)isoindolin-2-yl)methyl)-4H-pyran-4-one (I) with p-toluenesulfonic acid, particularly in crystalline form.

The salt of compound (I) with p-toluenesulfonic acid has been found to exist in several crystalline forms. These crystalline forms have been characterized by X-ray powder diffraction (XRPD) studies.

The crystalline form 1 of the p-toluenesulfonic acid salt can be prepared, for example, by first dissolving compound (I) and p-toluenesulfonic acid monohydrate, for example in equivalent molar amounts, in a suitable solvent. Suitable solvents include, for example, acetonitrile, 1-propanol, 2-butanol and ethanol. The mixture can be heated, for example, to the refluxing temperature. The obtained solution is stirred and slowly cooled, for example, to room temperature. The precipitated crystalline salt can then be isolated, for example, by filtering and dried at reduced pressure, for example, under vacuum at about 20-40° C., for example, for about 1-6 hours. Alternatively, crystalline form 1 can be prepared by adding to the solution of compound (I) and p-toluenesulfonic acid monohydrate, for example, in acetonitrile, a suitable antisolvent, for example tert-butyl methyl ether. The formed crystalline solid may then be isolated, for example, by filtering and dried as described above.

Accordingly, in one aspect, the present disclosure provides crystalline form 1 of the salt of compound (I) with p-toluenesulfonic acid having an X-ray powder diffraction pattern comprising characteristic peaks at about 4.4, 7.6, 11.5, 16.4, 17.7, 20.2 and 24.6 degrees 2-theta.

In yet another aspect, the present disclosure provides crystalline form 1 of the salt of compound (I) with p-toluenesulfonic acid having an X-ray powder diffraction pattern comprising characteristic peaks at about 4.4, 7.6, 8.8, 11.5, 13.2, 15.1, 16.4, 17.7, 19.7, 20.2 and 24.6 degrees 2-theta.

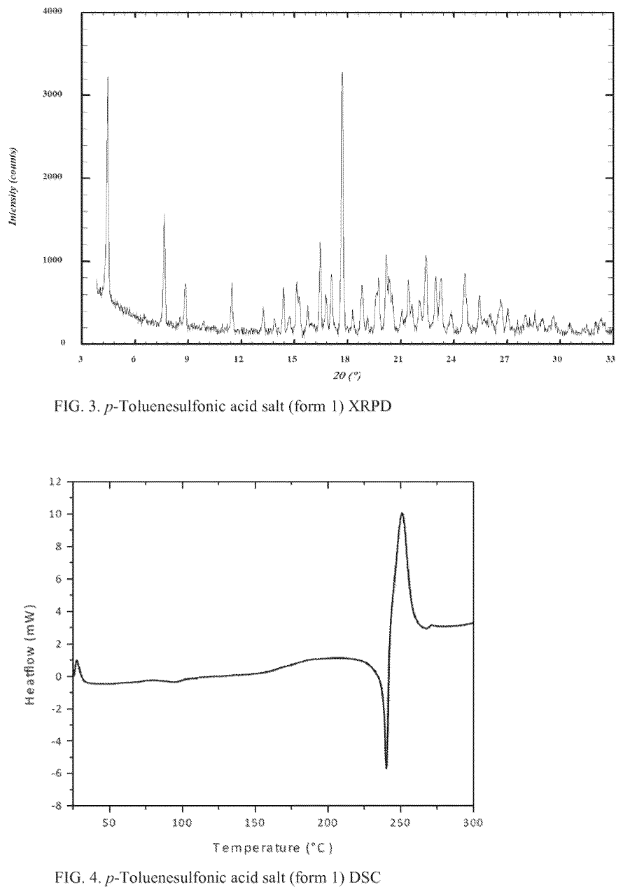

In a further aspect, the crystalline form 1 of the salt of compound (I) with p-toluenesulfonic acid is further characterized by an X-ray powder diffraction pattern as depicted in FIG. 3. In still another aspect, said crystalline form 1 is in the form of an anhydrate.

The crystalline form 2 of the p-toluenesulfonic acid salt can be prepared, for example, by dissolving compound (I) and p-toluenesulfonic acid monohydrate, for example, in equivalent molar amounts, in a suitable solvent. Suitable solvents include, for example, a mixture of acetonitrile and water. The mixture can be heated, for example, to refluxing temperature. The solution can then be concentrated, for example, under vacuum at about room temperature until the product is precipitated. The formed crystalline solid may then be isolated, for example, by filtering. The crystalline product can be dried at reduced pressure, for example, under vacuum at about 20-40° C., for example, for about 10-60 hours.

In a preferred method, crystalline form 2 is prepared in high polymorphic purity and in consistent manner by first dissolving compound (I) in a mixture of acetonitrile and water, where the amount of water is from about 5% to about 15%, preferably from 6% to 12%, per volume of the mixture. The amount of compound (I) is suitably from about 5 g to about 15 g, preferably from about 9 g to 13 g, for example, about 11 g, per 100 ml of the solvent. If desired, activated carbon, celite and/or triamine metal scavenger may also be added. The mixture may be heated, for example, to a temperature of about 50-80° C., more specifically to about 70-80° C. The mixture can be thereafter filtered. To the filtrate is then added p-toluenesulfonic acid monohydrate, for example, in about equivalent molar amount, under heating at a temperature of about 50-80° C., more specifically at about 70° C. The mixture may then be cooled to about 60° C. and seeded, if desired. The mixture can then be stirred in this temperature for about 1 h, followed by cooling, for example to about 0-20° C., more specifically to about 5° C., to precipitate the product. The crystalline form 2 can be washed, for example, with water, and dried at about 20-60° C., preferably at about 25-35° C., for example at about 30° C., under reduced pressure. It has been found that higher drying temperatures induce some transformation of crystalline form 2 into crystalline form 1. It was also found that existence of organic solvents in the wet mass before drying reduces the temperature where the crystalline transformation occurs. Therefore, using water as the washing liquid and using low drying temperatures are preferred.

Accordingly, in one aspect, the present disclosure provides crystalline form 2 of the salt of compound (I) with p-toluenesulfonic acid having an X-ray powder diffraction pattern comprising characteristic peaks at about 4.4, 6.5, 13.0, 18.8, 20.1 and 22.4 degrees 2-theta.

In yet another aspect, the present disclosure provides crystalline form 2 of the salt of compound (I) with p-toluenesulfonic acid having an X-ray powder diffraction pattern comprising characteristic peaks at about 4.4, 6.5, 8.3, 8.7, 10.9, 11.2, 13.0, 18.8, 19.6, 20.1, 21.7 and 22.4 degrees 2-theta.

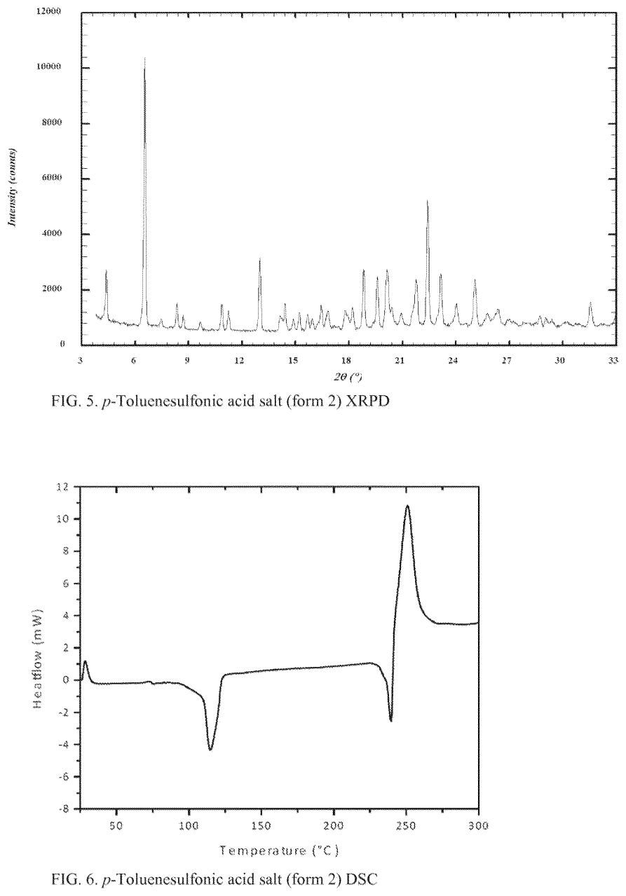

In a further aspect, the crystalline form 2 of the salt of compound (I) with p-toluenesulfonic acid is further characterized by an X-ray powder diffraction pattern as depicted in FIG. 5. In still another aspect, said crystalline form 2 is in the form of a hydrate.

The crystalline form 3 of the p-toluenesulfonic acid salt can be prepared, for example, by drying crystalline form 2 of p-toluenesulfonic acid salt of compound (I), for example, under vacuum at about 105° C. for about 24 h, followed by stirring the obtained product in a suitable solvent such as pentane to obtain a partially dissolved slurry. The slurry can be concentrated, for example by boiling at atmospheric pressure, to obtain the crystalline product.

Accordingly, in one aspect, the present disclosure provides crystalline form 3 of the salt of compound (I) with p-toluenesulfonic acid having an X-ray powder diffraction pattern comprising characteristic peaks at about 4.2, 8.6, 11.0, 12.9 and 17.2 degrees 2-theta.

In yet another aspect, the present disclosure provides crystalline form 3 of the salt of compound (I) with p-toluenesulfonic acid having an X-ray powder diffraction pattern comprising characteristic peaks at about 4.2, 8.6, 11.0, 12.9, 17.2, 18.6, 20.2 and 26.8 degrees 2-theta.

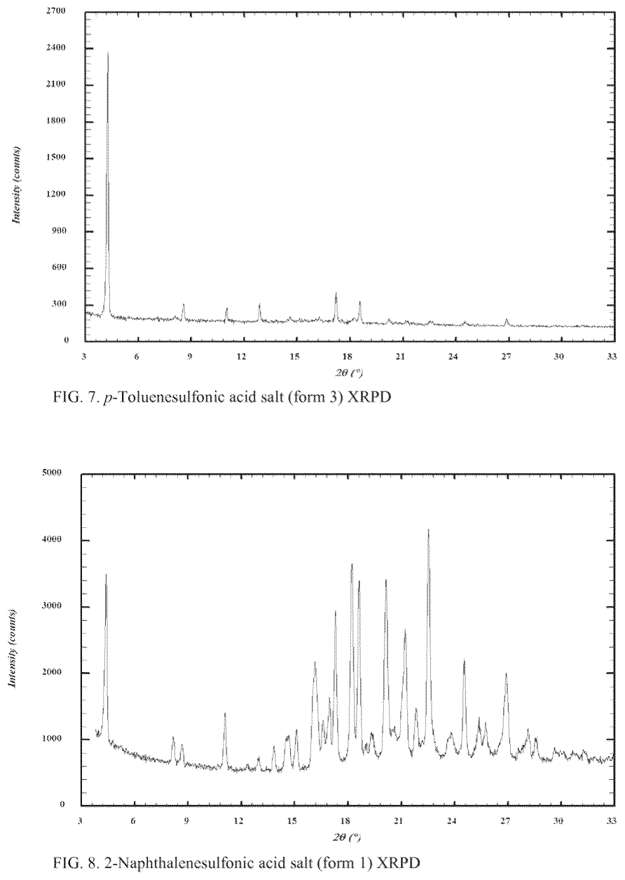

In a further aspect, the crystalline form 3 of the salt of compound (I) with p-toluenesulfonic acid is further characterized by an X-ray powder diffraction pattern as depicted in FIG. 7. In still another aspect, said crystalline form 3 is in the form of an anhydrate.

Salt with 2-Naphthalenesulfonic Acid

In another aspect, the present disclosure provides a salt of 5-((1-(methyl-sulfonyl)piperidin-4-yl)methoxy)-2-((5-(trifluoromethyl)isoindolin-2-yl)methyl)-4H-pyran-4-one (I) with 2-naphthalenesulfonic acid, particularly in crystalline form.

The salt of compound (I) with 2-naphthalenesulfonic acid appears to exist in a single crystalline form, here named as crystalline form 1. No other crystalline forms have been found for 2-naphthalenesulfonic acid salt.

The crystalline form 1 of 2-naphthalenesulfonic acid salt of compound (I) can be prepared, for example, by dissolving compound (I) and 2-naphthalenesulfonic acid, for example, in equivalent molar amounts, in a suitable solvent such as a mixture of acetonitrile and water. The mixture can be heated, for example, to a temperature of about 50-70° C. The obtained solution can then be cooled and concentrated, for example, under vacuum, to obtain the crystalline product. The product can be dried at reduced pressure, for example, under vacuum at about 20-40° C., for example, for about 10-60 hours.

Alternatively, the crystalline form 1 of the 2-naphthalenesulfonic acid salt of compound (I) can be prepared, by first dissolving compound (I) in a suitable solvent such as acetonitrile, suitably under heating at about 50-70° C. Then about a molar equivalent of 2-naphthalenesulfonic acid is added, suitably dropwise under stirring, to give a suspension. The mixture can then be cooled, for example to about room temperature, and the solids isolated for example by filtering. The crystalline product can be washed with ethanol and dried for example in reduced pressure at about 40° C.

The crystalline form 1 of the salt of compound (I) with 2-naphthalenesulfonic acid has been characterized by X-ray powder diffraction (XRPD) studies.

Accordingly, in one aspect, the present disclosure provides crystalline form 1 of the salt of compound (I) with 2-naphthalenesulfonic acid having an X-ray powder diffraction pattern comprising characteristic peaks at about 4.4, 11.1, 18.2, 18.6, 20.1 and 22.5 degrees 2-theta.

In yet another aspect, the present disclosure provides crystalline form 1 of the salt of compound (I) with 2-naphthalenesulfonic acid having an X-ray powder diffraction pattern comprising characteristic peaks at about 4.4, 8.1, 8.6, 11.1, 15.1, 16.2, 17.3, 18.2, 18.6, 20.1, 21.2 and 22.5 degrees 2-theta.

In a further aspect, the crystalline form 1 of the salt of compound (I) with 2-naphthalenesulfonic acid is further characterized by an X-ray powder diffraction pattern as depicted in FIG. 8.

Salt with Hydrobromic Acid

In another aspect, the present disclosure provides a salt of 5-((1-(methyl-sulfonyl)piperidin-4-yl)methoxy)-2-((5-(trifluoromethyl)isoindolin-2-yl)methyl)-4H-pyran-4-one (I) with hydrobromic acid, particularly in crystalline form.

The salt of compound (I) with hydrobromic acid appears to exist in a single crystalline form, here named as crystalline form 1. No other crystalline forms have been found for hydrobromic acid salt.

The crystalline form 1 of the hydrobromic acid salt can be prepared, for example, by dissolving about a molar equivalent of compound (I) and hydrobromic acid, for example, 48% aqueous hydrobromic acid, in a suitable solvent such as a mixture of methanol and water. The mixture can be heated, for example to about 50° C. The obtained solution is stirred and slowly cooled, for example to about 0-10° C. The precipitated crystalline salt can then be isolated, for example, by filtering, washed, and dried at reduced pressure, for example, under vacuum at about room temperature for about 5-10 h.

Alternatively, the crystalline form 1 of hydrobromic acid salt can be prepared by first dissolving compound (I) in a suitable solvent such as acetonitrile, suitably under heating at about 50-70° C. Then about a molar equivalent of 48% aqueous hydrobromic acid is added, suitably dropwise under stirring, to give a suspension. The mixture can then be cooled, for example to about 0-5° C., and the solids isolated, for example, by filtering. The crystalline product can be washed with ethanol and dried, for example, in reduced pressure at about 40° C.

The crystalline form 1 of the salt of compound (I) with hydrobromic acid has been characterized by X-ray powder diffraction (XRPD) studies.

Accordingly, in one aspect, the present disclosure provides crystalline form 1 of the salt of compound (I) with hydrobromic acid having an X-ray powder diffraction pattern comprising characteristic peaks at about 4.7, 7.0, 11.5, 18.5, 20.8 and 22.3 degrees 2-theta.

In yet another aspect, the present disclosure provides crystalline form 1 of the salt of compound (I) with hydrobromic acid having an X-ray powder diffraction pattern comprising characteristic peaks at about 4.7, 7.0, 11.5, 16.2, 17.2, 18.5, 20.2, 20.8, 22.3 and 22.5 degrees 2-theta.

In a further aspect, the crystalline form 1 of the salt of compound (I) with hydrobromic acid is further characterized by an X-ray powder diffraction pattern as depicted in FIG. 10.

Salt with Hydrochloric Acid

In another aspect, the present disclosure provides a salt of 5-((1-(methyl-sulfonyl)piperidin-4-yl)methoxy)-2-((5-(trifluoromethyl)isoindolin-2-yl)methyl)-4H-pyran-4-one (I) with hydrochloric acid, particularly in crystalline form.

The salt of compound (I) with hydrochloric acid appears to exist in a single crystalline form, here named as crystalline form 1.

The crystalline form 1 of the hydrochloric acid salt of compound (I) can be prepared, for example, by dissolving about a molar equivalent of compound (I) and hydrochloric acid, for example 35% aqueous hydrochloric acid, in suitable solvent such as acetonitrile. Thereafter, a suitable antisolvent such as tert-butyl methyl ether is added under stirring and the mixture is cooled, for example, to about 0-10° C. The precipitated crystalline salt can then be isolated, for example, by filtering, washed, and dried at reduced pressure, for example, under vacuum at about 20-40° C., for example, for about 1-5 hours.

Alternatively, the crystalline form 1 of hydrochloric acid salt can be prepared by mixing compound (I) and hydrochloric acid, for example 30% aqueous hydrochloric acid, in a suitable solvent such as methanol, suitably under heating at about 50-70° C. The mixture can then be cooled, for example to about 0-5° C., and water can be added during cooling. The solids formed are isolated, for example, by filtering. The crystalline product can be washed with ethanol and dried for example in reduced pressure at about 40° C.

The crystalline form of the salt of compound (I) with hydrochloric acid has been characterized by X-ray powder diffraction (XRPD) studies.

Thus, in one aspect, the present disclosure provides crystalline form 1 of the salt of compound (I) with hydrochloric acid having an X-ray powder diffraction pattern comprising characteristic peaks at about 6.8, 13.6, 15.9, 17.7, 18.2, 18.9 and 22.7 degrees 2-theta.

In yet another aspect, the present disclosure provides crystalline form 1 of the salt of compound (I) with hydrochloric acid having an X-ray powder diffraction pattern comprising characteristic peaks at about 6.8, 9.1, 11.3, 13.6, 15.9, 17.7, 18.2, 18.9, 19.9, 22.4 and 22.7 degrees 2-theta.

In a further aspect, the crystalline form 1 of the salt of compound (I) with hydrochloric acid is further characterized by an X-ray powder diffraction pattern as depicted in FIG. 12.

Salt with Methanesulfonic Acid

In another aspect, the present disclosure provides a salt of 5-((1-(methyl-sulfonyl)piperidin-4-yl)methoxy)-2-((5-(trifluoromethyl)isoindolin-2-yl)methyl)-4H-pyran-4-one (I) with methanesulfonic acid, particularly in crystalline form.

The salt of compound (I) with methanesulfonic acid appears to exist in a single crystalline form, here named as crystalline form 1.

The crystalline form 1 of the methanesulfonic acid salt can be prepared, for example, by dissolving about a molar equivalent of compound (I) and methanesulfonic acid in a suitable solvent such as tetrahydrofuran, suitably under heating, for example, at about 50-60° C. Thereafter, a suitable antisolvent such as hexane is added under stirring and the mixture is cooled, for example, to about 0-10° C. The precipitated crystalline salt can then be isolated, for example, by filtering, washed, and dried at reduced pressure, for example, under vacuum at about 20-40° C., for example, for about 1-5 hours.

Alternatively, the crystalline form 1 of the methanesulfonic acid salt can be prepared by first dissolving compound (I) in a suitable solvent such as acetonitrile, suitably under heating at about 50-60° C. Then about a molar equivalent of methanesulfonic acid is added, suitably dropwise under stirring, to give a suspension. The mixture can then be cooled, for example, to about 0-5° C., and the solids isolated for example by filtering. The crystalline product can be washed with ethanol and dried for example in reduced pressure at about 40° C.

The crystalline form 1 of the salt of compound (I) with methanesulfonic acid has been characterized by X-ray powder diffraction (XRPD) studies.

Thus, in one aspect, the present disclosure provides crystalline form 1 of the salt of compound (I) with methanesulfonic acid having an X-ray powder diffraction pattern comprising characteristic peaks at about 5.2, 16.7, 17.5, 21.1, 21.9 and 24.4 degrees 2-theta.

In yet another aspect, the present disclosure provides crystalline form 1 of the salt of compound (I) with methanesulfonic acid having an X-ray powder diffraction pattern comprising characteristic peaks at about 5.2, 12.4, 14.6, 15.6, 16.7, 17.5, 19.4, 21.1, 21.9 and 24.4 degrees 2-theta.

In a further aspect, the crystalline form 1 of the salt of compound (I) with methanesulfonic acid is further characterized by an X-ray powder diffraction pattern as depicted in FIG. 14.

Salt with Phosphoric Acid

In another aspect, the present disclosure provides a salt of 5-((1-(methyl-sulfonyl)piperidin-4-yl)methoxy)-2-((5-(trifluoromethyl)isoindolin-2-yl)methyl)-4H-pyran-4-one (I) with phosphoric acid, particularly in crystalline form.

The salt of compound (I) with phosphoric acid appears to exist in a single crystalline form, here named as crystalline form 1.

The crystalline form 1 of the phosphoric acid salt can be prepared, for example, by first dissolving compound (I) in a suitable solvent such as tetrahydro-furan, suitably under heating, for example, at about 50-60° C. Then about a molar equivalent of phosphoric acid, for example, 85% aqueous phosphoric acid, is added under stirring. Thereafter, a suitable antisolvent such as hexane is added under stirring and the mixture is cooled, for example, to about 0-10° C. The precipitated crystalline salt can then be isolated, for example by filtering, washed, and dried at reduced pressure, for example, under vacuum at about 20-40° C., for example, for about 1-5 hours.

The crystalline form 1 of the salt of compound (I) with phosphoric acid has been characterized by X-ray powder diffraction (XRPD) studies.

Thus, in one aspect, the present disclosure provides crystalline form 1 of the salt of compound (I) with phosphoric acid having an X-ray powder diffraction pattern comprising characteristic peaks at about 4.3, 6.7, 8.6, 14.5, 17.1, 19.3 and 21.2 degrees 2-theta.

In yet another aspect, the present disclosure provides crystalline form 1 of the salt of compound (I) with phosphoric acid having an X-ray powder diffraction pattern comprising characteristic peaks at about 4.3, 6.7, 8.6, 12.4, 14.5, 15.4, 16.5, 17.0, 17.1, 19.3, 20.2 and 21.2 degrees 2-theta.

In a further aspect, the crystalline form 1 of the salt of compound (I) with phosphoric acid is further characterized by an X-ray powder diffraction pattern as depicted in FIG. 16.

Salt with Maleic Acid

In another aspect, the present disclosure provides a salt of 5-((1-(methyl-sulfonyl)piperidin-4-yl)methoxy)-2-((5-(trifluoromethyl)isoindolin-2-yl)methyl)-4H-pyran-4-one (I) with maleic acid, particularly in crystalline form.

The crystalline form 1 of the maleic acid salt can be prepared, for example, by dissolving about a molar equivalent of compound (I) and maleic acid in a suitable solvent such as a mixture of acetonitrile and water, suitably under heating, for example, at about 50-60° C. Then about a molar equivalent of maleic acid, is added under stirring. The mixture is then cooled and the solvent evaporated, for example, under vacuum at about 40° C., to obtain the crystalline product.

Alternatively, the maleic acid salt can be prepared by first adding compound (I) in a suitable solvent such as isopropanol, suitably under heating at about 60-70° C. Then about a molar equivalent of maleic acid is added, suitably dropwise under stirring, to give a suspension. The mixture can then be cooled, for example, to about 0-5° C., and the solids isolated for example by filtering. The crystalline product can be dried, for example, under reduced pressure at about 40° C.

The crystalline form 1 of the salt of compound (I) with maleic acid has been characterized by X-ray powder diffraction (XRPD) studies.

Thus, in one aspect, the present disclosure provides crystalline form 1 of the salt of compound (I) with maleic acid having an X-ray powder diffraction pattern comprising characteristic peaks at about 12.4, 17.0, 18.6, 19.3, 20.8 and 23.9 degrees 2-theta.

In yet another aspect, the present disclosure provides crystalline form 1 of the salt of compound (I) with maleic acid having an X-ray powder diffraction pattern comprising characteristic peaks at about 12.4, 17.0, 18.6, 19.3, 20.0, 20.7, 20.8 and 23.9 degrees 2-theta.

In a further aspect, the crystalline form 1 of the salt of compound (I) with maleic acid is further characterized by an X-ray powder diffraction pattern as depicted in FIG. 18.

Salt with Benzenesulfonic Acid

In another aspect, the present disclosure provides a salt of 5-((1-(methyl-sulfonyl)piperidin-4-yl)methoxy)-2-((5-(trifluoromethyl)isoindolin-2-yl)methyl)-4H-pyran-4-one (I) with benzenesulfonic acid, particularly in crystalline form.

The crystalline form 1 of benzenesulfonic acid salt can be prepared, for example, by dissolving about a molar equivalent of compound (I) and benzene-sulfonic acid in a suitable solvent such as a mixture of methanol and water, suitably under heating, for example, at about 50-60° C. The solution is then cooled and the solvent evaporated, for example, under vacuum at about 40° C., to obtain the crystalline product.

Alternatively, benzenesulfonic acid salt of compound (I) can be prepared by first adding compound (I) in a suitable solvent such as acetonitrile, suitably under heating at about 60-70° C. Then about a molar equivalent of benzenesulfonic acid is added, suitably dropwise under stirring, to give a suspension. The mixture can then be cooled, for example to about 0-5° C., and the solids isolated for example by filtering. The crystalline product can be washed in ethanol, dried for example under reduced pressure at about 40° C.

The crystalline form 1 of the salt of compound (I) with benzenesulfonic acid has been characterized by X-ray powder diffraction (XRPD) studies.

Thus, in one aspect, the present disclosure provides crystalline form 1 of the salt of compound (I) with benzenesulfonic acid having an X-ray powder diffraction pattern comprising characteristic peaks at about 4.5, 6.7, 11.1 and 13.2 degrees 2-theta.

In yet another aspect, the present disclosure provides crystalline form 1 of the salt of compound (I) with benzenesulfonic acid having an X-ray powder diffraction pattern comprising characteristic peaks at about 4.5, 6.7, 8.9, 11.1, 13.2 and 20.1 degrees 2-theta.

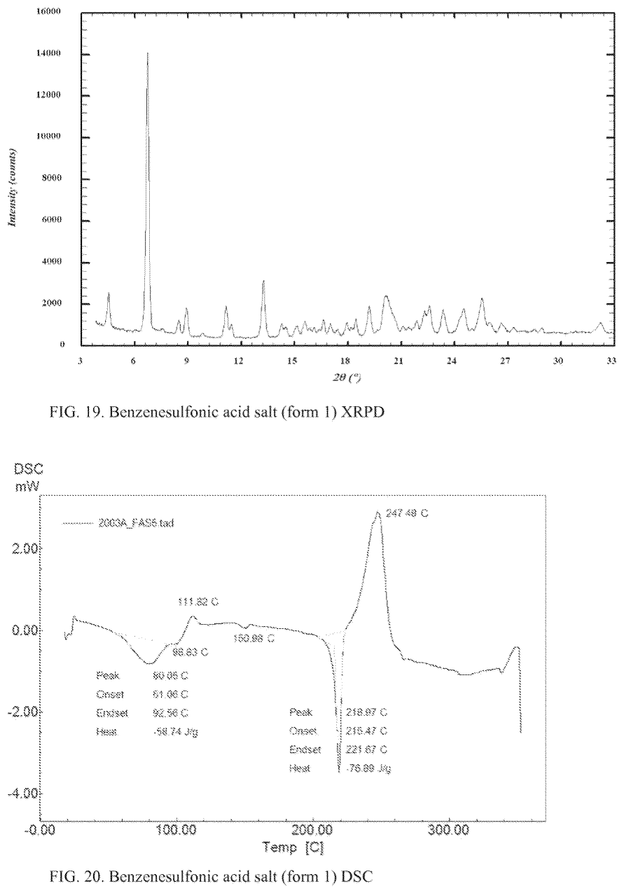

In a further aspect, the crystalline form 1 of the salt of compound (I) with benzenesulfonic acid is further characterized by an X-ray powder diffraction pattern as depicted in FIG. 19.

Salt with Oxalic Acid

In another aspect, the present disclosure provides a salt of 5-((1-(methyl-sulfonyl)piperidin-4-yl)methoxy)-2-((5-(trifluoromethyl)isoindolin-2-yl)methyl)-4H-pyran-4-one (I) with oxalic acid, particularly in crystalline form.

The salt with oxalic acid has been found to exist in several crystalline forms, named here as crystalline forms 1 to 4. These crystalline forms have been characterized by X-ray powder diffraction (XRPD) studies.

The crystalline form 1 of the oxalic acid salt can be prepared, for example, by first dissolving compound (I) and oxalic acid, for example, in equivalent molar amounts, in a suitable solvent such as methanol. The mixture can be heated, for example, at about 50-60° C. Thereafter, a suitable antisolvent such as tert-butyl methyl ether is added under stirring and the mixture is cooled, for example, to about 0-10° C. The precipitated crystalline salt can then be isolated, for example, by filtering and dried at reduced pressure, for example, under vacuum at about 20-40° C., for example, for about 1-6 hours.

The crystalline form 1 of the salt of compound (I) with oxalic acid has been characterized by X-ray powder diffraction (XRPD) studies.

Accordingly, in one aspect, the present disclosure provides crystalline form 1 of the salt of compound (I) with oxalic acid having an X-ray powder diffraction pattern comprising characteristic peaks at about 5.9, 11.8, 16.4, 18.6, 19.2 and 20.8 degrees 2-theta.

In yet another aspect, the present disclosure provides crystalline form 1 of the salt of compound (I) with oxalic acid having an X-ray powder diffraction pattern comprising characteristic peaks at about 5.9, 6.4, 11.8, 13.1, 14.4, 16.4, 18.6, 19.2, 20.8, 21.5, 22.0 and 23.7 degrees 2-theta.

In a further aspect, the crystalline form 1 of the salt of compound (I) with oxalic acid is further characterized by an X-ray powder diffraction pattern as depicted in FIG. 21.

The crystalline form 2 of the oxalic acid salt can be prepared, for example, by first dissolving compound (I) and oxalic acid, for example, in equivalent molar amounts, in a suitable solvent such as a mixture of acetonitrile and water, suitably under heating, for example, at about 50-60° C. The obtained solution can then be cooled and concentrated, for example, under vacuum, to precipitate the crystalline product. The product can be isolated, for example, by filtering, and dried at reduced pressure, for example, at vacuum at about 20-30° C., for example, for about 10-60 hours.

The crystalline form 2 of the salt of compound (I) with oxalic acid has been characterized by X-ray powder diffraction (XRPD) studies.

Accordingly, in one aspect, the present disclosure provides crystalline form 2 of the salt of compound (I) with oxalic acid having an X-ray powder diffraction pattern comprising characteristic peaks at about 5.0, 7.8, 10.0, 15.6, 20.8 and 25.0 degrees 2-theta.

In yet another aspect, the present disclosure provides crystalline form 2 of the salt of compound (I) with oxalic acid having an X-ray powder diffraction pattern comprising characteristic peaks at about 5.0, 7.8, 10.0, 11.6, 12.4, 13.8, 15.6, 17.3, 18.4, 20.8 and 25.0 degrees 2-theta.

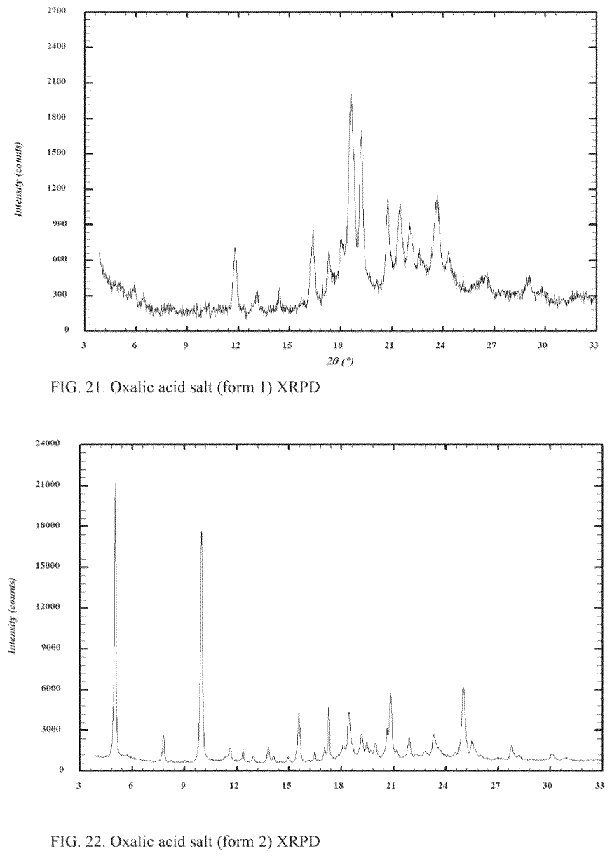

In a further aspect, the crystalline form 2 of the salt of compound (I) with oxalic acid is further characterized by an X-ray powder diffraction pattern as depicted in FIG. 22.

The crystalline form 3 of the oxalic acid salt can be prepared, for example, by first dissolving compound (I) and oxalic acid, for example in equivalent molar amounts, in a suitable solvent such as tetrahydrofuran. The mixture can be heated, for example, at about 50-60° C. Thereafter, a suitable antisolvent such as hexane is added under stirring and the mixture is cooled, for example, to about 0-10° C. The precipitated crystalline salt can then be isolated, for example, by filtering and dried at reduced pressure, for example under vacuum at about 20-40° C., for example, for about 1-6 hours.

The crystalline form 3 of the salt of compound (I) with oxalic acid has been characterized by X-ray powder diffraction (XRPD) studies.

Accordingly, in one aspect, the present disclosure provides crystalline form 3 of the salt of compound (I) with oxalic acid having an X-ray powder diffraction pattern comprising characteristic peaks at about 7.9, 13.3, 16.5, 17.3, 20.3 and 20.8 degrees 2-theta.

In yet another aspect, the present disclosure provides crystalline form 3 of the salt of compound (I) with oxalic acid having an X-ray powder diffraction pattern comprising characteristic peaks at about 7.9, 13.3, 16.5, 17.0, 17.1, 17.3, 17.9, 19.6, 20.3, 20.8, 21.9 and 25.7 degrees 2-theta.

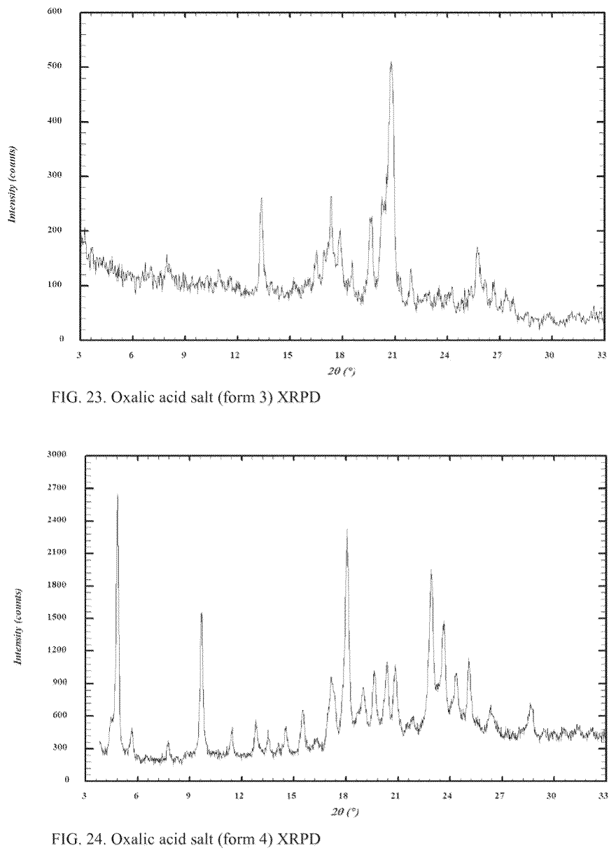

In a further aspect, the crystalline form 3 of the salt of compound (I) with oxalic acid is further characterized by an X-ray powder diffraction pattern as depicted in FIG. 23.

The crystalline form 4 of the oxalic acid salt can be prepared, for example, by first dissolving compound (I) and oxalic acid, for example in equivalent molar amounts, in a suitable solvent such as acetonitrile. Thereafter, a suitable antisolvent such as tert-butyl methyl ether is added under stirring and the mixture is cooled, for example, to about 0-10° C. The precipitated crystalline salt can then be isolated, for example, by filtering and dried at reduced pressure, for example, under vacuum at about 20-40° C., for example, for about 1-6 hours.

The crystalline form 4 of the salt of compound (I) with oxalic acid has been characterized by X-ray powder diffraction (XRPD) studies.

Accordingly, in one aspect, the present disclosure provides crystalline form 4 of the salt of compound (I) with oxalic acid having an X-ray powder diffraction pattern comprising characteristic peaks at about 4.4, 4.8, 5.7, 9.7, 18.1 and 22.9 degrees 2-theta.

In yet another aspect, the present disclosure provides crystalline form 4 of the salt of compound (I) with oxalic acid having an X-ray powder diffraction pattern comprising characteristic peaks at about 4.4, 4.8, 5.7, 7.8, 9.7, 11.5, 12.8, 15.5, 18.1, 22.9 and 23.7 degrees 2-theta.

In a further aspect, the crystalline form 4 of the salt of compound (I) with oxalic acid is further characterized by an X-ray powder diffraction pattern as depicted in FIG. 24.

The above XRPD peak positions refer to values, when measured using CuKα radiation (λ=1.5418 Å). It will be recognized by the skilled person that the X-ray powder diffraction pattern peak positions referred to herein can be subject to variations of ±0.2, ±0.15 or ±0.1 degrees 2-theta according to various factors such as temperature, sample handling and instrumentation used.

The above crystalline salts of compound (I) can be formulated into pharmaceutical dosage forms such as tablets, capsules, granules, powders or suspensions together with excipients known in the art.

Thus, in one aspect, the present disclosure provides a pharmaceutical composition comprising any of the above salts of compound (I) or crystalline forms thereof together with one or more excipients, particularly in the form of a tablet or a capsule.

In another aspect, the present disclosure provides substantially pure crystalline forms of the salts of compound (I), as disclosed above, wherein at least 90%, preferably at least 95%, more preferably at least 98%, per weight of the salt of compound (I) is present in said crystalline form.

The invention is further illustrated by the following non-limiting examples.

### Analytical Methods

XRPD measurements were performed with the X-ray powder diffractometer Bruker D8 Advance at room temperature, using a copper filled X-ray tube (40 kV×40 mA) as the X-ray source, CuKα (λ=1.5418 Å), a 0.6 mm divergence slit, 2.5° Soller slits on both the primary and secondary beams and the 1-dimensional LynxEye detector (with aperture angle of 2.91593°). Data collection was done in the range of 3.8-33° 2θ with 0.02° increments, at a scan speed of 0.3°/s.

Differential scanning calorimetry (DSC) was carried out on a Shimadzu DSC-60 calorimeter in the range of 20-300° C. with a constant heating rate of 10° C./min in crimped aluminium sample cell with nitrogen flow of 60 ml/min.

Melting point was determined by observing the phase change during hot stage microscopy in open chamber with heating rate of 10° C./min.

Single-crystal diffraction data were collected on a Rigaku Oxford Diffraction SuperNova dual-wavelength diffractometer with the operating mirror monochromated Cu Kα (λ=1.5418 Å) or Mo Kα radiation mode (λ=0.7107 Å). X-ray data collection was monitored, and all data were corrected for Lorentzian, polarization, and absorption effects using the CrysAlisPro program. The Olex₂ program was used for the crystal structure solution and refinement, SHELXS97 for structure solution, and SHELXL for full-matrix least-squares refinement on F².

### Example 1. Amorphous Form of Compound (I)

Concentrated solution (20.83 mg/ml) of compound (I) in 2-butanone was prepared by stirring at room temperature. The solvent was then removed at room temperature under vacuum. The resulting solids were analyzed by XRPD (data not shown). The procedure produced amorphous form of compound (I).

### Example 2. Crystalline Form 1 of Compound (I)

Solution (6.7 mg/ml) of compound (I) in 1-propanol was prepared by heating the mixture of compound (I) and 1-propanol at 50° C. until complete dissolution. The solution was cooled at room temperature followed by ageing at 5° C. for 24 h. After the ageing, solvent evaporation was carried out at vacuum at 40° C. The resulting solids were analyzed by XRPD. The procedure produced crystalline form 1 of compound (I) (Table 1). The X-ray powder diffraction pattern of crystalline form 1 of compound (I) is depicted in FIG. 1 and the differential scanning calorimetry (DSC) thermogram in FIG. 2.

TABLE 1
X-ray powder reflections (up to 33° 2θ) and intensities
(normalized) of crystalline form 1 of compound (I). The value
2θ [°] represents the diffraction angle in degrees
and the value d [Å] represents the specified distances
in Å between the lattice planes.

| 2θ [°] | d [Å] | I/Iₒ [%] |
|---|---|---|
| 8.27 | 10.69 | 21 |
| 9.75 | 9.07 | 3 |
| 12.38 | 7.14 | 90 |
| 14.59 | 6.07 | 31 |
| 15.45 | 5.73 | 45 |
| 16.50 | 5.37 | 60 |
| 17.11 | 5.18 | 100 |
| 17.34 | 5.11 | 45 |
| 17.86 | 4.96 | 29 |
| 18.11 | 4.89 | 6 |
| 19.02 | 4.66 | 17 |
| 19.46 | 4.56 | 12 |
| 20.16 | 4.40 | 8 |
| 20.60 | 4.31 | 98 |
| 21.30 | 4.17 | 7 |
| 21.85 | 4.06 | 40 |
| 22.85 | 3.89 | 27 |
| 23.11 | 3.85 | 5 |
| 23.73 | 3.75 | 2 |
| 24.15 | 3.68 | 6 |
| 24.58 | 3.62 | 5 |
| 24.84 | 3.58 | 23 |
| 25.75 | 3.46 | 59 |
| 26.04 | 3.42 | 4 |
| 29.02 | 3.07 | 14 |
| 31.47 | 2.84 | 3 |

### Example 3. p-Toluenesulfonic Acid Salt Crystalline Form 1

300.7 mg of compound (I) was mixed with 12 ml of acetonitrile followed by heating the mixture at 50° C. until complete dissolution. Then 119.3 mg of p-toluene-sulfonic acid monohydrate (1 molar equivalent) dissolved in 4 ml of acetonitrile was added under stirring. Subsequently, 16 ml of tert-butyl methyl ether was added followed by ageing the mixture at 5° C. for 24 h. The formed precipitate was decanted and air-dried followed by drying at vacuum (200 mbar) at 30° C. for 2 h. The resulting solids were analyzed by XRPD. The procedure produced crystalline form 1 of p-toluenesulfonic acid salt of compound (I) (Table 2). The X-ray powder diffraction pattern of crystalline form 1 of p-toluenesulfonic acid salt is depicted in FIG. 3 and the differential scanning calorimetry (DSC) thermogram in FIG. 4. ¹H NMR (600 MHZ, DMSO-d₆): δ 1.26-1.35 (m, 2H), 1.81-1.87 (m, 2H), 1.83-1.89 (m, 1H), 2.27 (s, 3H), 2.73 (ddd, 2H), 2.86 (s, 3H), 3.59 (ddd, 2H), 3.74 (d, 2H), 4.69 (s, 2H), 4.82 (br s, 4H), 6.70 (s, 1H), 7.07 (d, 2H), 7.42 (d, 2H), 7.62 (d, 1H), 7.74 (d, 1H), 7.80 (s, 1H), 8.19 (s, 1H), 11.50 (br s, 1H).

TABLE 2
X-ray powder reflections (up to 33° 2θ) and intensities (normalized)
of p-toluenesulfonic acid salt crystalline form 1. The value 2θ
[°] represents the diffraction angle in degrees and the value
d [Å] represents the specified distances in Å between the lattice planes.

| 2θ [°] | d [Å] | I/Iₒ [%] | 2θ [°] | d [Å] | I/Iₒ [%] |
|---|---|---|---|---|---|
| 4.44 | 19.90 | 86 | 8.82 | 10.02 | 16 |
| 7.64 | 11.57 | 43 | 11.45 | 7.72 | 19 |
| 13.23 | 6.69 | 9 | 21.05 | 4.22 | 7 |
| 13.85 | 6.39 | 5 | 21.42 | 4.15 | 18 |
| 14.34 | 6.17 | 18 | 21.56 | 4.12 | 10 |
| 14.71 | 6.02 | 6 | 22.05 | 4.03 | 10 |
| 15.08 | 5.87 | 20 | 22.42 | 3.96 | 28 |
| 15.26 | 5.80 | 14 | 22.96 | 3.87 | 20 |
| 15.74 | 5.62 | 10 | 23.26 | 3.82 | 20 |
| 16.44 | 5.39 | 35 | 23.83 | 3.73 | 6 |
| 16.77 | 5.28 | 13 | 24.62 | 3.61 | 23 |
| 17.07 | 5.19 | 22 | 25.44 | 3.50 | 14 |
| 17.66 | 5.02 | 100 | 26.02 | 3.42 | 6 |
| 18.28 | 4.85 | 9 | 26.64 | 3.34 | 11 |
| 18.80 | 4.72 | 19 | 27.05 | 3.29 | 8 |
| 19.11 | 4.64 | 7 | 27.97 | 3.19 | 7 |
| 19.57 | 4.53 | 15 | 28.57 | 3.12 | 7 |
| 19.72 | 4.50 | 21 | 29.00 | 3.08 | 5 |
| 20.16 | 4.40 | 29 | 29.67 | 3.01 | 6 |
| 20.31 | 4.37 | 21 | 30.51 | 2.93 | 4 |
| 20.49 | 4.33 | 14 | | | |

### Example 3. p-Toluenesulfonic Acid Salt Crystalline Form 2

To a reactor under nitrogen was added acetonitrile (65 ml), water (5.2 ml), compound (I) (10 g), activated carbon (1.0 g), celite (1.5 g) and triamine metal scavenger (0.5 g). The mixture was heated to 70±5° C. and agitated for 1.0±0.5 h. The solids were filtered and washed with acetonitrile (19 ml) and water (0.9 ml). In a separate vessel, p-toluenesulfonic acid monohydrate (5.5 g) was dissolved in water (6 ml) and acetonitrile (9 ml). The p-toluenesulfonic acid solution was added to the mixture at 68±3° C. After the addition, the mixture was cooled to 62±3° C. and seeded. The suspension was stirred at this temperature for about 1 h, cooled to 50±3° C. over about 2 h, heated to 60° C. over about 1 h followed by agitation for about 1 h, cooled to 45° C. over about 3 h, heated to 55° C. over about 1 h, cooled to 5° C. over about 3 h followed by stirring for at least 1 h. The product is collected and washed with water (30 ml) and cold isopropanol (30 ml). The isolated wet solids were dried at 30° C. under vacuum to yield 12.0 g (86%) of white crystalline solids. The product was analysed by XRPD and was found to be crystalline form 2 of p-toluenesulfonic acid salt of compound (I) (Table 3). The X-ray powder diffraction pattern of form 2 is depicted in FIG. 5 and the differential scanning calorimetry (DSC) thermogram in FIG. 6.

TABLE 3
X-ray powder reflections (up to 33° 2θ) and intensities (normalized)
of p-toluenesulfonic acid salt crystalline form 2. The value 2θ
[°] represents the diffraction angle in degrees and the value
d [Å] represents the specified distances in Å between the lattice planes.

| 2θ [°] | d [Å] | I/Iₒ [%] |
|---|---|---|
| 4.39 | 20.09 | 20 |
| 6.53 | 13.52 | 100 |
| 7.45 | 11.85 | 3 |
| 8.32 | 10.62 | 10 |
| 8.65 | 10.21 | 5 |
| 9.65 | 9.16 | 3 |
| 10.86 | 8.14 | 10 |
| 11.24 | 7.87 | 7 |
| 13.00 | 6.81 | 28 |
| 14.12 | 6.27 | 5 |
| 14.36 | 6.16 | 10 |
| 14.87 | 5.95 | 4 |
| 15.21 | 5.82 | 7 |
| 15.62 | 5.67 | 6 |
| 15.95 | 5.55 | 4 |
| 16.44 | 5.39 | 9 |
| 16.81 | 5.27 | 6 |
| 17.77 | 4.99 | 7 |
| 17.91 | 4.95 | 5 |
| 18.14 | 4.89 | 7 |
| 18.82 | 4.71 | 22 |
| 19.59 | 4.53 | 19 |
| 20.13 | 4.41 | 21 |
| 20.40 | 4.35 | 7 |
| 20.93 | 4.24 | 4 |
| 21.74 | 4.08 | 16 |
| 22.40 | 3.97 | 46 |
| 23.15 | 3.84 | 19 |
| 24.02 | 3.70 | 8 |
| 25.07 | 3.55 | 17 |
| 25.77 | 3.45 | 5 |
| 26.37 | 3.38 | 6 |
| 28.71 | 3.11 | 4 |
| 29.09 | 3.07 | 3 |
| 31.47 | 2.84 | 8 |

### Example 4. p-Toluenesulfonic Acid Salt Crystalline Form 2 (Alternative Method)

150.2 mg of compound (I) was mixed with 10 ml of acetonitrile: water mixture (50:50 by volume) followed by vortexing until complete dissolution. Then 59.54 mg of p-toluenesulfonic acid monohydrate (1 molar equivalent) dissolved in 2 ml of acetonitrile:water mixture (50:50 by volume) was added. The resulting mixture was heating at 50° C. for 30 min to obtain a clear solution. The still clear solution was placed under vacuum (200 mbar) at room temperature for 5 h to precipitate the solids. The mixture was decanted and the solids were dried at vacuum (200 mbar) first at room temperature for 40 h and then at 40° C. for 19 h. The resulting product was analyzed by XRPD. The procedure produced crystalline form 2 of p-toluene-sulfonic acid salt of compound (I).

### Example 5. p-Toluenesulfonic Acid Salt Crystalline Form 3

24.4 mg of p-toluenesulfonic acid salt of compound (I) obtained by drying crystalline form 2 of p-toluenesulfonic acid salt of compound (I) under vacuum (150 mbar) at 105° C. for 24 h was mixed with 20 ml of pentane followed by vortexing to obtain a partially dissolved slurry. The slurry was concentrated by boiling at 37-40° C. in atmospheric pressure. The resulting solids were analyzed by XRPD. The procedure produced crystalline form 3 of p-toluenesulfonic acid salt of compound (I) (Table 4). The X-ray powder diffraction pattern of crystalline form 3 of p-toluene-sulfonic acid salt is depicted in FIG. 7.

TABLE 4
X-ray powder reflections (up to 33° 2θ) and intensities (normalized)
of p-toluenesulfonic acid salt crystalline form 3. The value 2θ
[°] represents the diffraction angle in degrees and the value
d [Å] represents the specified distances in Å between the lattice planes.

| 2θ [°] | d [Å] | I/Iₒ [%] |
|---|---|---|
| 4.22 | 20.90 | 100 |
| 8.56 | 10.32 | 6 |
| 10.98 | 8.05 | 5 |
| 12.86 | 6.88 | 8 |
| 17.20 | 5.15 | 11 |
| 18.55 | 4.78 | 8 |
| 20.17 | 4.40 | 2 |
| 26.84 | 3.32 | 2 |

### Example 6. Amorphous p-Toluenesulfonic Acid Salt

Solution (2 mg/ml) of p-toluenesulfonic acid salt of compound (I) in a 20:80 mixture of 2,2,2-trifluoroethanol: water was prepared by stirring the mixture at room temperature to obtain a clear solution. Thereafter the solution was freeze-dried at −36° C. followed by solvent removal at vacuum for 2 days. The resulting solids were analyzed by XRPD (data not shown). The procedure produced amorphous form of p-toluenesulfonic acid salt of compound (I).

### Example 7. 2-Naphthalenesulfonic Acid Salt Crystalline Form 1

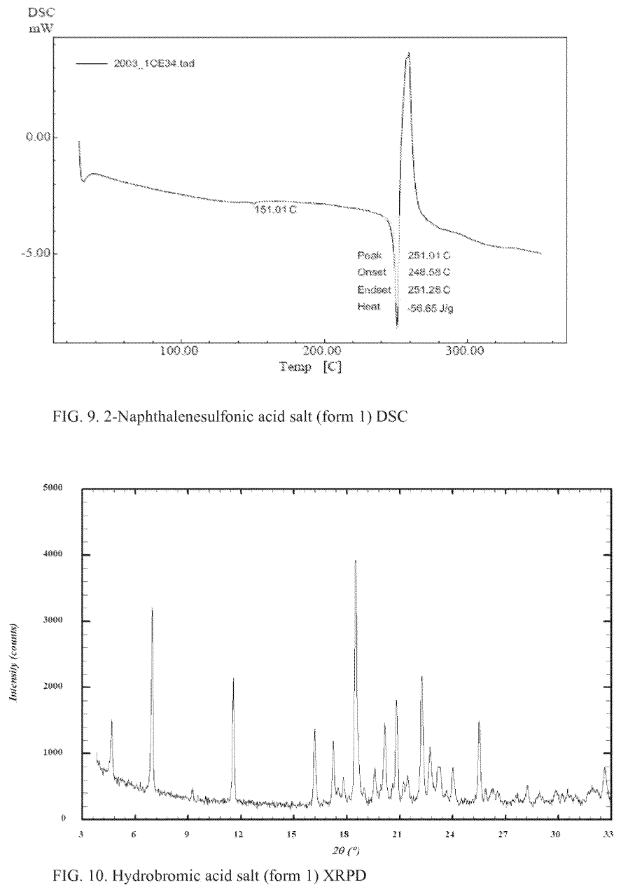

150.7 mg of compound (I) was mixed with 10 ml of acetonitrile: water mixture (50:50 by volume) followed by vortexing until complete dissolution. Subsequently, 65.51 mg of 2-naphthalenesulfonic acid (1 molar equivalent) dissolved in acetonitrile:water mixture (50:50 by volume) was added under stirring. The resulting mixture was heated at 50° C. for 30 min for obtaining a clear solution. The solution was cooled at room temperature during 2 h followed by ageing at 5° C. for 24 h. The still clear solution was placed under vacuum (200 mbar) at room temperature for 12 h to form the precipitate. The precipitate was dried at vacuum first at room temperature for 32 h and then at 40° C. for 30 h. The resulting solids were analyzed by XRPD. The procedure produced crystalline form 1 of 2-naphthalenesulfonic acid salt of compound (I) (Table 5). The X-ray powder diffraction pattern of crystalline form 1 of 2-naphthalenesulfonic acid salt is depicted in FIG. 8 and the differential scanning calorimetry (DSC) thermogram in FIG. 9.

TABLE 5
X-ray powder reflections (up to 33° 2θ) and intensities (normalized)
of 2-naphthalenesulfonic acid salt crystalline form 1. The value 2θ
[°] represents the diffraction angle in degrees and the value
d [Å] represents the specified distances in Å between the lattice planes.

| 2θ [°] | d [Å] | I/Iₒ [%] |
|---|---|---|
| 4.36 | 20.24 | 81 |
| 8.12 | 10.88 | 12 |
| 8.64 | 10.23 | 10 |
| 11.07 | 7.98 | 27 |
| 12.32 | 7.18 | 3 |
| 12.94 | 6.83 | 5 |
| 13.83 | 6.40 | 13 |
| 14.56 | 6.08 | 15 |
| 14.66 | 6.04 | 17 |
| 15.09 | 5.87 | 17 |
| 16.15 | 5.48 | 48 |
| 16.59 | 5.34 | 20 |
| 16.92 | 5.24 | 29 |
| 17.25 | 5.14 | 69 |
| 18.21 | 4.87 | 92 |
| 18.62 | 4.76 | 84 |
| 19.35 | 4.58 | 11 |
| 20.13 | 4.41 | 79 |
| 21.21 | 4.19 | 53 |
| 21.83 | 4.07 | 17 |
| 22.52 | 3.94 | 100 |
| 23.77 | 3.74 | 11 |
| 24.51 | 3.63 | 44 |
| 25.39 | 3.50 | 14 |
| 25.74 | 3.46 | 13 |
| 26.90 | 3.31 | 38 |
| 28.08 | 3.18 | 13 |
| 28.56 | 3.12 | 10 |

### Example 8. 2-Naphthalenesulfonic Acid Salt Crystalline Form 1 (Alternative Method)

To a round bottom flask under nitrogen was added compound (I) (10.0 g) followed by acetonitrile (60 ml). The mixture was heated to 65° C. upon which a clear solution was obtained. 2-Naphthalenesulfonic acid (4.71 g, 1.1 molar equivalent) was added dropwise to give a thick suspension. The suspension was diluted with acetonitrile (30 ml) to improve stirrability. The mass was allowed to cool to room temperature. The product was collected by filtration and washed with ethanol (40 ml). After drying in vacuum at 40° C., 13.4 g (94%) of crystalline form 1 was obtained. 1H NMR (400 MHZ, DMSO-d₆): δ 8.21 (s, 1H), 8.13 (d, J=1.6 Hz, 1H), 7.99-7.93 (m, 1H), 7.93-7.87 (m, 1H), 7.85 (d, J=8.5 Hz, 1H), 7.81 (s, 1H), 7.74 (dd, J=8.1, 1.7 Hz, 1H), 7.69 (dd, J=8.5, 1.7 Hz, 1H), 7.62 (d, J=8.0 Hz, 1H), 7.56-7.49 (m, 2H), 6.66 (s, 1H), 4.77 (s, 4H), 4.61 (s, 2H), 3.72 (d, J=5.9 Hz, 2H), 3.59 (dt, J=12.3, 3.3 Hz, 2H), 2.86 (s, 3H), 2.72 (td, J=12.1, 2.4 Hz, 2H), 1.97-1.77 (m, 3H), 1.30 (qd, J=12.8, 4.0 Hz, 2H).

### Example 9. Hydrobromic Acid Salt Crystalline Form 1

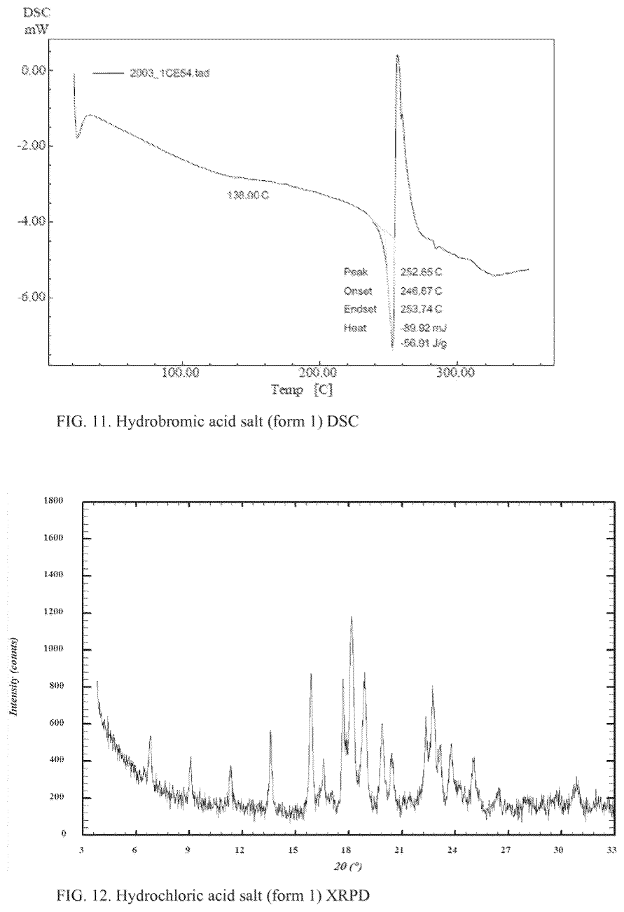

150.4 mg of compound (I) was mixed with 10 ml of methanol: water mixture (90:10 by volume) followed by heating at 50° C. until complete dissolution. Then 35.63 μl of 47% aqueous hydrobromic acid (1 molar equivalent) was added under stirring. The resulting mixture was heated at 50° C. for 30 min to obtain a clear solution. The mixture was then cooled at room temperature during 2 h followed by ageing at 5° C. for 24 h. The formed precipitate was decanted and dried at vacuum (200 mbar) at room temperature for 5 h. The resulting solids were analyzed by XRPD. The procedure produced crystalline form 1 of hydrobromic acid salt of compound (I) (Table 6). The X-ray powder diffraction pattern of crystalline form 1 of hydrobromic acid salt is depicted in FIG. 10 and the differential scanning calorimetry (DSC) thermogram in FIG. 11.

TABLE 6
X-ray powder reflections (up to 33° 2θ) and intensities (normalized)
of hydrobromic acid salt crystalline form 1. The value 2θ [°]
represents the diffraction angle in degrees and the value d [Å]
represents the specified distances in Å between the lattice planes.

| 2θ [°] | d [Å] | I/Iₒ [%] |
|---|---|---|
| 4.65 | 19.01 | 23 |
| 6.95 | 12.70 | 74 |
| 9.24 | 9.57 | 4 |
| 11.54 | 7.66 | 50 |
| 16.19 | 5.47 | 31 |
| 17.22 | 5.15 | 25 |
| 17.81 | 4.98 | 9 |
| 18.50 | 4.79 | 100 |
| 18.98 | 4.67 | 5 |
| 19.57 | 4.53 | 11 |
| 20.15 | 4.40 | 31 |
| 20.80 | 4.27 | 35 |
| 21.22 | 4.18 | 6 |
| 21.44 | 4.14 | 9 |
| 22.25 | 3.99 | 50 |
| 22.71 | 3.91 | 21 |
| 23.23 | 3.83 | 12 |
| 23.65 | 3.76 | 3 |
| 24.02 | 3.70 | 12 |
| 25.51 | 3.49 | 33 |
| 25.88 | 3.44 | 4 |
| 26.28 | 3.39 | 4 |
| 26.60 | 3.35 | 4 |
| 28.24 | 3.16 | 7 |
| 28.90 | 3.09 | 4 |
| 29.84 | 2.99 | 4 |
| 32.64 | 2.74 | 14 |

### Example 10. Hydrobromic Acid Salt Crystalline Form 1 (Alternative Method)

To a round bottom flask under nitrogen was added compound (I) (10.0 g) followed by acetonitrile (60 ml). The mixture was heated to 55° C. upon which a clear solution was obtained. 48% aqueous hydrobromic acid (3.46 g, 1.1 molar equivalent) was added dropwise to give a thick suspension. The suspension was diluted with acetonitrile (15 ml) to improve stirrability. The mass was allowed to cool to room temperature and then in an ice batch. The product was collected by filtration and washed with ethanol (40 ml). After drying in vacuum at 40° C. 11.1 g (95%) of crystalline form 1 was obtained. 1H NMR (400 MHZ, DMSO-d₆) δ: 8.25 (s, 1H), 7.84 (s, 1H), 7.76 (dd, J=8.1, 1.7 Hz, 1H), 7.65 (d, J=8.0 Hz, 1H), 6.70 (s, 1H), 4.79 (s, 4H), 4.67 (s, 2H), 3.75 (d, J=6.0 Hz, 2H), 3.66-3.48 (m, 2H), 2.87 (s, 3H), 2.74 (td, J=12.0, 2.3 Hz, 2H), 1.85 (dt, J=12.7, 3.0 Hz, 3H), 1.31 (qd, J=12.5, 12.0, 3.9 Hz, 2H).

### Example 11. Hydrochloric Acid Salt Crystalline Form 1

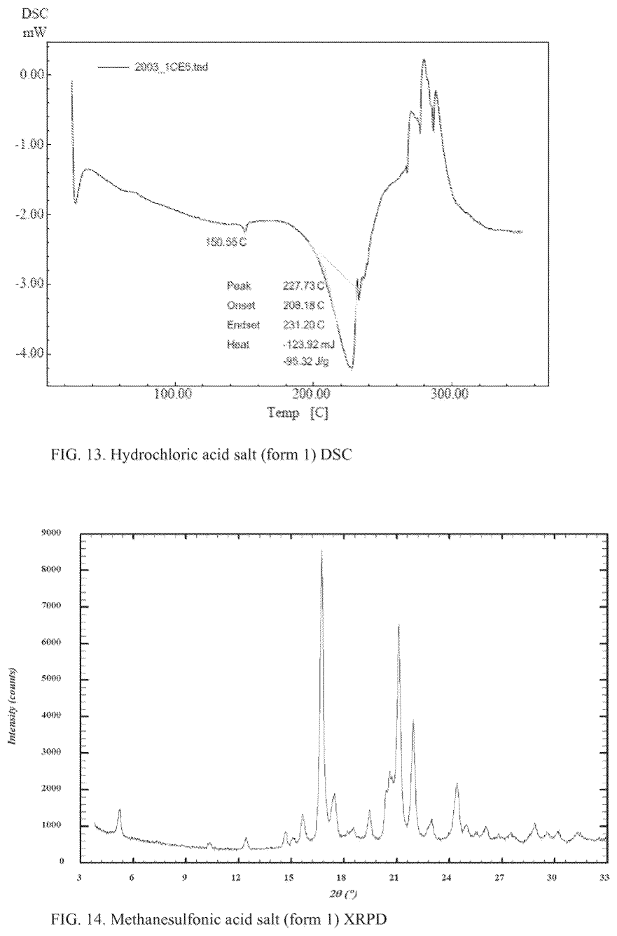

300 mg of compound (I) was mixed with 15 ml of acetonitrile followed by vortexing until complete dissolution. Then 53.53 μl of 35% aqueous hydrochloric acid (1 molar equivalent) was added under stirring. Subsequently, 15 ml of tert-butyl methyl ether was added under stirring and the resulting mixture was aged at 5° C. for 72 h. The formed precipitate was decanted and dried at vacuum (200 mbar) at 30° C. for 2 h. The resulting solids were analyzed by XRPD. The procedure produced crystalline form 1 of hydrochloric acid salt of compound (I) (Table 7). The X-ray powder diffraction pattern of crystalline form 1 of hydrochloric acid salt is depicted in FIG. 12 and the differential scanning calorimetry (DSC) thermogram in FIG. 13.

TABLE 7
X-ray powder reflections (up to 33° 2θ) and intensities
(normalized) of hydrochloric acid salt crystalline form 1.
The value 2θ [°] represents the diffraction angle
in degrees and the value d [Å] represents the specified
distances in Å between the lattice planes.

| 2θ [°] | d [Å] | I/Iₒ [%] | 2θ [°] | d [Å] | I/Iₒ [%] |
|---|---|---|---|---|---|
| 6.80 | 12.99 | 24 | 17.67 | 5.02 | 69 |
| 9.08 | 9.73 | 22 | 18.17 | 4.88 | 100 |
| 11.33 | 7.80 | 22 | 18.88 | 4.70 | 69 |
| 13.58 | 6.52 | 42 | 19.87 | 4.46 | 45 |
| 15.87 | 5.58 | 72 | 20.40 | 4.35 | 30 |
| 16.58 | 5.34 | 27 | 22.35 | 3.97 | 49 |
| 22.69 | 3.92 | 63 | 25.05 | 3.55 | 27 |
| 22.82 | 3.89 | 56 | 26.45 | 3.37 | 10 |
| 23.13 | 3.84 | 33 | 30.85 | 2.90 | 14 |
| 23.75 | 3.74 | 34 | | | |

### Example 12. Hydrochloric Acid Salt Crystalline Form 1 (Alternative Method)

To a round bottom flask under nitrogen was added compound (I) (10.0 g) followed by methanol (70 ml) and 30% hydrochloric acid (2.75 g, 1.1 molar equivalent). The mixture was heated to 65° C. The suspension was diluted with methanol (30 ml) to improve stirrability. The mass was allowed to cool. During cooling 20 ml of water was added to the mixture. After reaching room temperature, the mass was further cooled in an ice batch. The product was collected by filtration and washed with ethanol (40 ml). After drying in vacuum at 40° C. 10.4 g (96%) of crystalline form 1 was obtained. 1H NMR (400 MHZ, DMSO-d₆) δ 8.22 (s, 1H), 7.81 (s, 1H), 7.78-7.71 (m, 1H), 7.62 (d, J=8.1 Hz, 1H), 6.74 (s, 1H), 4.73 (s, 5H), 4.58 (s, 2H), 3.74 (d, J=6.0 Hz, 2H), 3.63-3.54 (m, 3H), 3.52 (s, 6H), 2.86 (s, 3H), 2.73 (td, J=12.0, 2.4 Hz, 2H), 1.96-1.74 (m, 3H), 1.30 (qd, J=12.9, 12.5, 4.1 Hz, 2H).

### Example 13. Methanesulfonic Acid Salt Crystalline Form 1

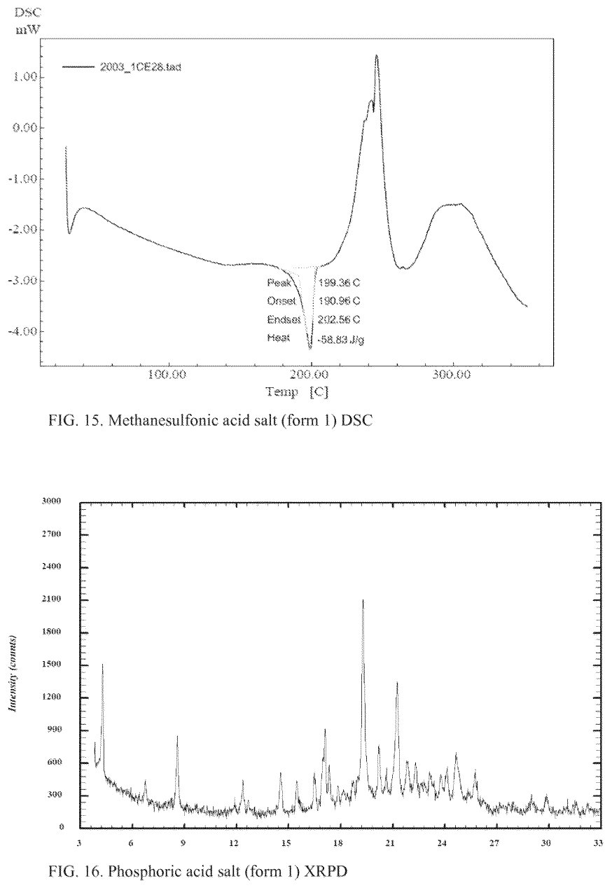

300 mg of compound (I) was mixed with 16 ml of tetrahydrofuran followed by heating at 50° C. until complete dissolution. Then 40.86 μl of methanesulfonic acid (1 molar equivalent) was added. Subsequently, 16 ml of hexane was added under stirring followed by ageing at 5° C. for 24 h. The precipitate was decanted and air-dried followed by drying at vacuum (200 mbar) at 30° C. for 2 h. The resulting solids were analyzed by XRPD. The procedure produced crystalline form 1 of methanesulfonic acid salt of compound (I) (Table 8). The X-ray powder diffraction pattern of crystalline form 1 of methanesulfonic acid salt is depicted in FIG. 14 and the differential scanning calorimetry (DSC) thermogram in FIG. 15.

TABLE 8
X-ray powder reflections (up to 33° 2θ) and intensities
(normalized) of methanesulfonic acid salt crystalline form 1.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 5.21 | 16.95 | 10 |
| 10.29 | 8.59 | 2 |
| 12.43 | 7.12 | 4 |
| 14.64 | 6.05 | 5 |
| 15.15 | 5.84 | 2 |
| 15.64 | 5.66 | 9 |
| 16.73 | 5.30 | 100 |
| 17.45 | 5.08 | 14 |
| 18.47 | 4.80 | 3 |
| 19.44 | 4.56 | 10 |
| 20.38 | 4.35 | 3 |
| 20.61 | 4.31 | 8 |
| 21.11 | 4.20 | 65 |
| 21.93 | 4.05 | 37 |
| 22.96 | 3.87 | 6 |
| 24.42 | 3.64 | 17 |
| 24.91 | 3.57 | 3 |
| 25.50 | 3.49 | 1 |
| 26.09 | 3.41 | 4 |
| 27.49 | 3.24 | 3 |
| 28.85 | 3.09 | 5 |
| 29.55 | 3.02 | 2 |
| 30.14 | 2.96 | 4 |
| 31.32 | 2.85 | 3 |

### Example 14. Methanesulfonic Acid Salt Crystalline Form 1 (Alternative Method)

To a round bottom flask under nitrogen was added compound (I) (10.0 g) followed by acetonitrile (60 ml). The mixture was heated to 50° C. upon which a clear solution was obtained. Methanesulfonic acid (2.17 g, 1.1 molar equivalent) was added dropwise to give a thick suspension. The suspension was diluted with acetonitrile (15 ml) to improve stirrability. The mass was allowed to cool to room temperature and then in an ice batch. The product was collected by filtration and washed with ethanol (40 ml). After drying in vacuum at 40° C. 11.1 g (93%) of crystalline form 1 was obtained. 1H NMR (400 MHZ, DMSO-d₆) δ 8.24 (s, 1H), 7.83 (s, 1H), 7.79-7.72 (m, 1H), 7.64 (d, J=8.0 Hz, 1H), 6.67 (s, 1H), 4.77 (s, 4H), 4.61 (s, 2H), 3.74 (d, J=6.0 Hz, 2H), 3.65-3.52 (m, 2H), 2.86 (s, 3H), 2.73 (td, J=12.0, 2.3 Hz, 2H), 2.32 (s, 3H), 1.86 (dq, J=11.1, 5.2, 4.0 Hz, 3H), 1.31 (qd, J=12.6, 12.0, 4.0 Hz, 2H).

### Example 15. Phosphoric Acid Salt Crystalline Form 1

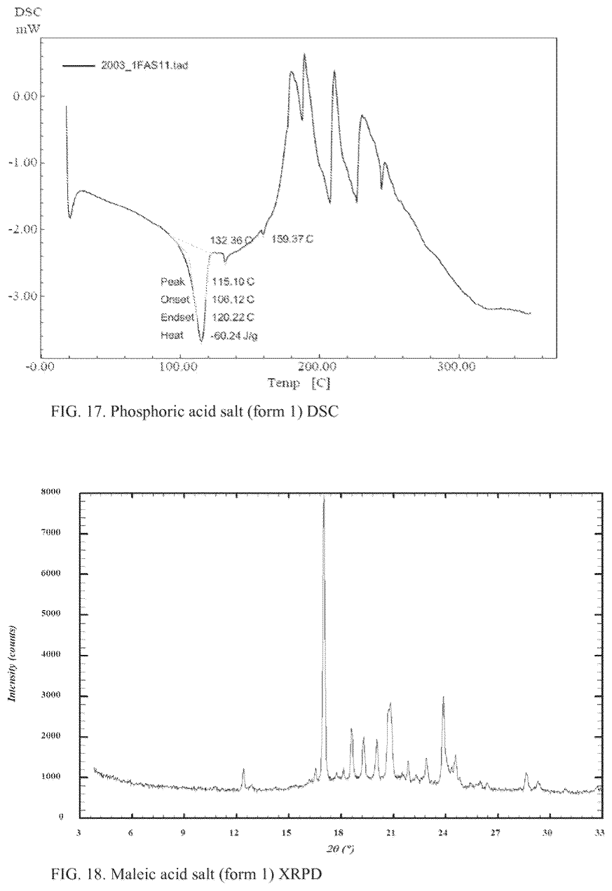

300.5 mg of compound (I) was mixed with 16 ml of tetrahydrofuran followed by heating at 50° C. until complete dissolution. Then 42.09 μl of 85% aqueous phosphoric acid was added (1 molar equivalent) under stirring. Subsequently, 16 ml of hexane was added under stirring and the resulting mixture was aged at 5° C. for 24 h. The precipitate was recovered by filtering and first air-dried followed by drying at vacuum (200 mbar) at 30° C. for 2 h. The resulting solids were analyzed by XRPD. The procedure produced crystalline form 1 of phosphoric acid salt of compound (I) (Table 9). The X-ray powder diffraction pattern of crystalline form 1 of phosphoric acid salt is depicted in FIG. 16 and the differential scanning calorimetry (DSC) thermogram in FIG. 17.

TABLE 9
X-ray powder reflections (up to 33° 2θ) and intensities (normalized)
of phosphoric acid salt crystalline form 1. The value 2θ [°]
represents the diffraction angle in degrees and the value d [Å]
represents the specified distances in Å between the lattice planes.

| 2θ [°] | d [Å] | I/Iₒ [%] |
|---|---|---|
| 4.27 | 20.67 | 55 |
| 6.72 | 13.14 | 11 |
| 8.58 | 10.30 | 37 |
| 11.87 | 7.45 | 6 |
| 12.37 | 7.15 | 15 |
| 12.68 | 6.98 | 7 |
| 14.54 | 6.09 | 19 |
| 15.44 | 5.74 | 15 |
| 16.46 | 5.38 | 18 |
| 16.95 | 5.23 | 25 |
| 17.09 | 5.18 | 38 |
| 17.29 | 5.12 | 19 |
| 17.83 | 4.97 | 8 |
| 18.15 | 4.88 | 5 |
| 18.69 | 4.74 | 8 |
| 19.28 | 4.60 | 100 |
| 20.21 | 4.39 | 25 |
| 20.68 | 4.29 | 13 |
| 21.24 | 4.18 | 56 |
| 21.86 | 4.06 | 15 |
| 22.30 | 3.98 | 14 |
| 23.11 | 3.85 | 10 |
| 23.38 | 3.80 | 7 |
| 23.77 | 3.74 | 12 |
| 24.17 | 3.68 | 16 |
| 24.63 | 3.61 | 24 |
| 25.32 | 3.51 | 8 |
| 25.74 | 3.46 | 18 |
| 28.98 | 3.08 | 6 |
| 29.84 | 2.99 | 7 |

### Example 16. Maleic Acid Salt Crystalline Form 1

300 mg of compound (I) was mixed with 20 ml of acetonitrile:water mixture (50:50 by volume) followed by heating at 50° C. until complete dissolution. Then 73 mg of maleic acid (1 molar equivalent) dissolved in 4 ml of acetonitrile: water mixture (50:50 by volume) was added. The resulting mixture was heated at 50° C. for 30 min to obtain a clear solution. The solution was cooled at room temperature during 2 h followed by ageing at 5° C. for 72 h. The still clear solution was placed under vacuum (200 mbar) at 40° C. for 40 h to evaporate the solvent. The resulting solids were analyzed by XRPD. The procedure produced crystalline form 1 (2a) of maleic acid salt of compound (I) (Table 10). The X-ray powder diffraction pattern of crystalline form 1 (2a) of maleic acid salt is depicted in FIG. 18.

TABLE 10
X-ray powder reflections (up to 33° 2θ) and intensities (normalized)
of maleic acid salt crystalline form 1 (2a). The value 2θ [°]
represents the diffraction angle in degrees and the value d [Å]
represents the specified distances in Å between the lattice planes.

| 2θ [°] | d [Å] | I/Iₒ [%] |
|---|---|---|
| 12.38 | 7.14 | 8 |
| 12.86 | 6.88 | 2 |
| 16.55 | 5.35 | 3 |
| 17.00 | 5.21 | 100 |
| 17.68 | 5.01 | 2 |
| 18.10 | 4.90 | 4 |
| 18.59 | 4.77 | 17 |
| 19.27 | 4.60 | 14 |
| 19.97 | 4.44 | 14 |
| 20.67 | 4.29 | 23 |
| 20.82 | 4.26 | 26 |
| 21.79 | 4.08 | 6 |
| 22.87 | 3.89 | 8 |
| 23.85 | 3.73 | 31 |
| 24.56 | 3.62 | 11 |
| 28.57 | 3.12 | 6 |
| 29.27 | 3.05 | 3 |

### Example 17. Maleic Acid Salt Crystalline Form 1 (Alternative Method)

To a round bottom flask under nitrogen was added compound (I) (3.0 g) followed by isopropanol (20 ml). The mixture was heated to 65° C. Maleic acid (0.79 g, 1.1 molar equivalent) was added slowly. During the addition the suspension became thinner, but thickened quickly as the product precipitated. The suspension was diluted with isopropanol (10 ml) and stirred at 65° C. for 1 h. The mass was allowed to cool to room temperature and then in an ice batch. The product was collected by filtration and washed with ice cold isopropanol (20 ml). After drying in vacuum at 40° C. 3.7 g (100%) of product was obtained. The resulting solids were analyzed by XRPD. The procedure produced maleic acid salt crystalline form 1 (2a). 1H NMR (400 MHZ, DMSO-d₆) δ 8.18 (s, 1H), 7.68 (d, J=1.8 Hz, 1H), 7.64-7.59 (m, 1H), 7.51 (d, J=7.9 Hz, 1H), 6.46 (s, 1H), 6.23 (s, 2H), 4.18 (s, 4H), 3.98 (s, 2H), 3.73 (d, J=6.0 Hz, 2H), 3.59 (dt, J=12.0, 3.4 Hz, 2H), 2.86 (s, 3H), 2.73 (td, J=12.0, 2.4 Hz, 2H), 1.85 (dp, J=8.4, 3.6, 2.8 Hz, 3H), 1.38-1.23 (m, 2H), 1.04 (d, J=6.1 Hz, 2H).

### Example 18. Benzenesulfonic Acid Salt Crystalline Form 1

149.9 mg of compound (I) was mixed with 10 ml of methanol: water mixture (90:10 by volume) followed by heating at 50° C. until complete dissolution. Then 49.8 mg of benzenesulfonic acid (1 molar equivalent) dissolved in 20 ml of methanol: water mixture (50:50 by volume) was added. The resulting mixture was heated at 50° C. for 30 min to obtain a clear solution. The solution was cooled at room temperature during 2 h followed by ageing at 5° C. for 24 h. The still clear solution was placed under vacuum (200 mbar) at 40° C. for 5 h to evaporate the solvent. The resulting solids were analyzed by XRPD. The procedure produced crystalline form 1 of benzenesulfonic acid salt of compound (I) (Table 11). The X-ray powder diffraction pattern of crystalline form 1 of benzenesulfonic acid salt is depicted in FIG. 19 and the differential scanning calorimetry (DSC) thermogram in FIG. 20.

TABLE 11
X-ray powder reflections (up to 33° 2θ) and intensities
(normalized) of benzenesulfonic acid salt crystalline form 1.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 4.51 | 19.56 | 13 |
| 6.71 | 13.15 | 100 |
| 8.45 | 10.45 | 4 |
| 8.93 | 9.89 | 10 |
| 11.14 | 7.94 | 12 |
| 11.43 | 7.73 | 4 |
| 13.24 | 6.68 | 20 |
| 14.27 | 6.20 | 5 |
| 14.53 | 6.09 | 3 |
| 15.13 | 5.85 | 3 |
| 15.58 | 5.68 | 5 |
| 16.09 | 5.50 | 2 |
| 16.59 | 5.34 | 5 |
| 16.96 | 5.22 | 3 |
| 17.40 | 5.09 | 2 |
| 17.94 | 4.94 | 5 |
| 18.44 | 4.81 | 6 |
| 19.13 | 4.64 | 10 |
| 20.16 | 4.40 | 12 |
| 21.05 | 4.22 | 1 |
| 21.87 | 4.06 | 3 |
| 22.30 | 3.98 | 7 |
| 22.59 | 3.93 | 9 |
| 23.35 | 3.81 | 8 |
| 24.47 | 3.63 | 8 |
| 25.55 | 3.48 | 11 |
| 25.98 | 3.43 | 3 |
| 26.61 | 3.35 | 3 |
| 27.27 | 3.27 | 2 |
| 32.24 | 2.77 | 3 |

### Example 19. Benzenesulfonic Acid Salt Crystalline Form 1 (Alternative Method)

To a round bottom flask under nitrogen was added compound (I) (3.0 g) followed by acetonitrile (20 ml). The mixture was heated to 65° C. Benzenesulfonic acid (1.07 g, 1.1 molar equivalent) was added slowly to give a thick suspension. The suspension was diluted with acetonitrile (20 ml). The mass was allowed to cool to room temperature and then in an ice batch. The product was collected by filtration and washed with ethanol (20 ml). After drying in vacuum at 40° C. 3.5 g (88%) of product was obtained. The resulting solids were analyzed by XRPD. The procedure produced benzenesulfonic acid salt crystalline form 1. ¹H NMR (400 MHZ, DMSO-d₆) δ 8.23 (s, 1H), 7.82 (s, 1H), 7.76 (dd, J=7.7, 1.4 Hz, 1H), 7.63 (d, J=8.0 Hz, 1H), 7.60-7.54 (m, 2H), 7.34-7.26 (m, 3H), 6.66 (s, 1H), 4.77 (s, 4H), 4.62 (s, 2H), 3.74 (d, J=6.0 Hz, 2H), 3.66-3.53 (m, 3H), 2.86 (s, 3H), 2.73 (td, J=12.0, 2.3 Hz, 2H), 1.85 (dt, J=12.3, 2.9 Hz, 3H), 1.30 (qd, J=13.0, 4.1 Hz, 2H).

### Example 20. Oxalic Acid Salt Crystalline Form 1

300.7 mg of compound (I) was mixed with 20 ml of methanol followed by heating at 50° C. until complete dissolution. Then 56.7 mg of oxalic acid (1 molar equivalent) dissolved in 4 ml of methanol was added under stirring. Subsequently, 24 ml of tert-butyl methyl ether was added under stirring followed by ageing the mixture at 5° C. for 24 h. The formed precipitate was recovered by filtering, first air-dried followed by drying under vacuum (200 mbar) at 30° C. for 2 h. The resulting solids were analyzed by XRPD. The procedure produced crystalline form 1 of oxalic acid salt of compound (I) (Table 12). The X-ray powder diffraction pattern of crystalline form 1 (1a) of oxalic acid salt is depicted in FIG. 21.

TABLE 12
X-ray powder reflections (up to 33° 2θ) and intensities
(normalized) of oxalic acid salt crystalline form 1. The value
2θ [°] represents the diffraction angle in degrees
and the value d [Å] represents the specified distances
in Å between the lattice planes.

| 2θ [°] | d [Å] | I/Iₒ [%] |
|---|---|---|
| 5.94 | 14.87 | 9 |
| 6.40 | 13.79 | 5 |
| 11.83 | 7.47 | 34 |
| 13.05 | 6.78 | 12 |
| 14.36 | 6.16 | 12 |
| 16.41 | 5.40 | 37 |
| 17.29 | 5.12 | 17 |
| 18.03 | 4.92 | 22 |
| 18.60 | 4.77 | 100 |
| 19.20 | 4.62 | 81 |
| 20.76 | 4.27 | 44 |
| 21.51 | 4.13 | 37 |
| 21.98 | 4.04 | 25 |
| 22.59 | 3.93 | 10 |
| 23.65 | 3.76 | 36 |
| 24.32 | 3.66 | 12 |

### Example 21. Oxalic Acid Salt Crystalline Form 2

150.5 mg of compound (I) was mixed with 10 ml of acetonitrile: water mixture (50:50 by volume) followed by vortexing until complete dissolution. Subsequently, 28.32 mg of oxalic acid (1 molar equivalent) dissolved in 2 ml of acetonitrile:water mixture (50:50 by volume) was added under stirring. The resulting mixture was heated at 50° C. for 30 min to obtain a clear solution. The solution was cooled at room temperature during 2 h and aged at 5° C. for 24 h. The still clear solution was placed under vacuum (200 mbar) at room temperature for 12 h to precipitate the solids. The formed precipitate was decanted and dried at vacuum (200 mbar) at room temperature for 32 h. The resulting solids were analyzed by XRPD. The procedure produced crystalline form 2 of oxalic acid salt of compound (I) (Table 13). The X-ray powder diffraction pattern of crystalline form 2 of oxalic acid salt is depicted in FIG. 22.

TABLE 13
X-ray powder reflections (up to 33° 2θ) and intensities
(normalized) of oxalic acid salt crystalline form 2. The value
2θ [°] represents the diffraction angle in degrees
and the value d [Å] represents the specified distances
in Å between the lattice planes.

| 2θ [°] | d [Å] | I/Iₒ [%] |
|---|---|---|
| 5.00 | 17.65 | 100 |
| 7.79 | 11.34 | 10 |
| 9.96 | 8.87 | 84 |
| 11.62 | 7.61 | 5 |
| 12.35 | 7.16 | 4 |
| 12.94 | 6.83 | 2 |
| 13.79 | 6.42 | 6 |
| 14.09 | 6.28 | 2 |
| 14.97 | 5.91 | 2 |
| 15.57 | 5.69 | 18 |
| 16.48 | 5.37 | 3 |
| 17.03 | 5.20 | 4 |
| 17.25 | 5.14 | 19 |
| 18.06 | 4.91 | 4 |
| 18.44 | 4.81 | 16 |
| 19.17 | 4.63 | 7 |
| 19.43 | 4.57 | 6 |
| 19.94 | 4.45 | 5 |
| 20.60 | 4.31 | 10 |
| 20.83 | 4.26 | 23 |
| 21.19 | 4.19 | 2 |
| 21.93 | 4.05 | 7 |
| 22.81 | 3.89 | 1 |
| 23.33 | 3.81 | 8 |
| 25.01 | 3.56 | 25 |
| 25.47 | 3.49 | 5 |
| 27.79 | 3.21 | 5 |
| 30.11 | 2.97 | 3 |

### Example 22. Oxalic Acid Salt Crystalline Form 3

300 mg of compound (I) was mixed with 16 ml of tetrahydrofuran followed by heating at 50° C. until complete dissolution. Then 56.6 mg of oxalic acid (1 molar equivalent) dissolved in 4 ml of tetrahydrofuran was added under stirring. Subsequently, 20 ml of hexane was added under stirring followed by ageing the mixture at 5° C. for 24 h. The formed precipitate was recovered by filtering, air-dried followed by drying under vacuum (200 mbar) at 30° C. for 2 h. The resulting solids were analyzed by XRPD. The procedure produced crystalline form 3 of oxalic acid salt of compound (I) (Table 14). The X-ray powder diffraction pattern of crystalline form 3 (3a) of oxalic acid salt is depicted in FIG. 23.

TABLE 14
X-ray powder reflections (up to 33° 2θ) and intensities
(normalized) of oxalic acid salt crystalline form 3. The value
2θ [°] represents the diffraction angle in degrees
and the value d [Å] represents the specified distances
in Å between the lattice planes.

| 2θ [°] | d [Å] | I/Iₒ [%] |
|---|---|---|
| 7.89 | 11.20 | 8 |
| 13.29 | 6.66 | 37 |
| 15.19 | 5.83 | 2 |
| 16.46 | 5.38 | 15 |
| 16.96 | 5.22 | 15 |
| 17.14 | 5.17 | 18 |
| 17.32 | 5.12 | 37 |
| 17.87 | 4.96 | 23 |
| 18.50 | 4.79 | 11 |
| 19.61 | 4.52 | 29 |
| 20.31 | 4.37 | 38 |
| 20.79 | 4.27 | 100 |
| 21.18 | 4.19 | 12 |
| 21.31 | 4.17 | 10 |
| 21.90 | 4.05 | 14 |
| 22.90 | 3.88 | 6 |
| 23.49 | 3.78 | 9 |
| 24.22 | 3.67 | 8 |
| 25.39 | 3.50 | 10 |
| 25.72 | 3.46 | 26 |
| 26.12 | 3.41 | 14 |
| 26.62 | 3.35 | 12 |
| 27.39 | 3.25 | 10 |
| 27.75 | 3.21 | 7 |

### Example 23. Oxalic Acid Salt Crystalline Form 4

300.3 mg of compound (I) was mixed with 12 ml of acetonitrile followed by vortexing until complete dissolution. Then 56.7 mg of oxalic acid (1 molar equivalent) dissolved in 4 ml of acetonitrile was added under stirring. Subsequently, 16 ml of tert-butyl methyl ether was added under stirring followed by ageing the mixture at 5° C. for 72 h. The formed precipitate was recovered by filtering, air-dried followed by drying under vacuum (200 mbar) at 30° C. for 2 h. The resulting solids were analyzed by XRPD. The procedure produced crystalline form 4 of oxalic acid salt of compound (I) (Table 15). The X-ray powder diffraction pattern of crystalline form 4 of oxalic acid salt is depicted in FIG. 24.

TABLE 15
X-ray powder reflections (up to 33° 2θ) and intensities
(normalized) of oxalic acid salt crystalline form 4. The value
2θ [°] represents the diffraction angle in degrees
and the value d [Å] represents the specified distances
in Å between the lattice planes.

| 2θ [°] | d [Å] | I/Iₒ [%] | 2θ [°] | d [Å] | I/Iₒ [%] |
|---|---|---|---|---|---|
| 4.39 | 20.09 | 14 | 9.67 | 9.14 | 56 |
| 4.83 | 18.27 | 100 | 11.46 | 7.72 | 12 |
| 5.66 | 15.61 | 11 | 12.77 | 6.93 | 13 |
| 7.76 | 11.38 | 8 | 13.47 | 6.57 | 10 |
| 14.03 | 6.31 | 4 | 20.86 | 4.26 | 23 |
| 14.54 | 6.09 | 11 | 21.79 | 4.08 | 5 |
| 15.50 | 5.71 | 15 | 22.91 | 3.88 | 51 |
| 16.32 | 5.43 | 4 | 23.66 | 3.76 | 29 |
| 17.16 | 5.16 | 24 | 24.36 | 3.65 | 16 |
| 18.05 | 4.91 | 76 | 25.08 | 3.55 | 24 |
| 18.99 | 4.67 | 14 | 26.28 | 3.39 | 10 |
| 19.63 | 4.52 | 19 | 28.57 | 3.12 | 12 |
| 20.34 | 4.36 | 24 | | | |

### Example 24. Oxalic acid salt (mixture of form 1 and 2)

To a round bottom flask under nitrogen was added Compound (I) (10.0 g) followed by acetonitrile (60 ml). The mixture was heated to 45° C. to give a clear solution. Oxalic acid (2.04 g, 1.1 molar equivalent) was added as a slurry in water (10 ml). When about half of the acid had been added the product precipitated. The mixture was further heated to 65° C. and water (5 ml) was added to produce a clear solution. The solution was allowed to cool and at about 47° C. the product precipitated again. The mixture was diluted with acetonitrile (20 ml) reheated to 55° C. and further diluted with acetonitrile (30 ml) and water (5 ml) after which the mixture became adequately stirrable. The mass was allowed to cool to room temperature and then in an ice batch. The product was collected by filtration and washed with ethanol (40 ml). After drying in vacuum at 40° C. 11.0 g (93%) of the product was obtained which was analyzed by XRPD. The procedure produced a mixture of crystalline form 1 and 2. ¹H NMR (400 MHZ, DMSO-d₆): δ 8.16 (s, 1H), 7.65 (d, J=1.9 Hz, 1H), 7.59 (dd, J=7.9, 1.7 Hz, 1H), 7.49 (d, J=7.9 Hz, 1H), 6.43 (s, 1H), 4.08 (s, 4H), 3.87 (s, 2H), 3.72 (d, J=6.0 Hz, 2H), 3.58 (dt, J=12.3, 3.4 Hz, 3H), 2.86 (s, 3H), 2.73 (td, J=12.0, 2.4 Hz, H), 1.92-1.76 (m, 3H), 1.37-1.22 (m, 2H).

### Example 25. Single Crystal X-Ray Diffraction Data of Crystalline Form 1 of Compound (I)

Unit cell parameters of crystalline form 1 of compound (I) were determined from single crystal X-ray diffraction data and are summarized below, T=293 (2) K, radiation wavelength MoKα (λ=0.71073 Å), structural formula C₂₂H₂₅F₃N₂O₅S.

| | | |
|---|---|---|
| Crystal system | Triclinic | |
| Space group | P-1 | |
| Unit cell dimensions | a = 5.05281(17) Å | α = 85.738(5)° |
| | b = 10.7047(9) Å | β = 86.683(3)° |
| | c = 21.6403(8) Å | γ = 76.833(5)° |
| Volume | V = 1135.57(11) Å³ | |
| Z | 2 | |
| Goodness-of-fit | 1.046 | |
| R factor | 0.0785 | |
| Morphology | Thin plate | |

### Example 26. Single Crystal X-Ray Diffraction Data of p-Toluenesulfonic Acid Salt Crystalline Form 1

Unit cell parameters of crystalline form 1 of p-toluenesulfonic acid salt of compound (I) were determined from single crystal X-ray diffraction data and are summarized below, T=293 (2) K, radiation wavelength MoKα (λ=0.71073 Å), structural formula C₂₂H₂₆F₃N₂O₅S·C₇H₇O₃S.

| | | |
|---|---|---|
| Crystal system | Monoclinic | |
| Space group | P2₁/c | |
| Unit cell dimensions | a = 6.4221(18) Å | α = 90° |
| | b = 12.162(4) Å | β = 93.06(3)° |
| | c = 40.297(12) Å | γ = 90° |
| Volume | V = 3143.0(16) Å³ | |
| Z | 4 | |
| Goodness-of-fit | 1.188 | |
| R factor | 0.2435 | |
| Morphology | Thin plate | |

### Example 27. Melting Points of the Salts of Compound (I)

Melting point of each salt of compound (I) was determined by observing the phase change during hot stage microscopy in open chamber. Heating rate 10° C./min. The results are shown in Table 16. A crystalline solid with high melting point tends to be easy to purify by re-crystallisation and stable on storage.

TABLE 16
Melting points of the salts of compound
(I) observed by hot stage microscopy

| Salt form of compound (I) | Melting point |
|---|---|
| 2-Naphthalenesulphonic acid salt (form 1) | 245° C. |
| Hydrobromic acid salt (form 1) | 240° C. |
| p-Toluenesulfonic acid salt (form 1) | 230° C. |
| p-Toluenesulfonic acid salt (form 2) | 230° C. |
| Methanesulfonic acid salt (form 1) | 170° C. |
| Hydrochloric acid salt (form 1) | 156° C. |
| Oxalic acid salt (form 2) | 148° C. |
| Benzenesulfonic acid salt (form 1) | 143° C. |
| Phosphoric acid salt (form 1) | 107° C. |

### Example 28. Stability of the Crystalline Forms of p-Toluenesulfonic Acid Salt in Different Water Activity Conditions

Stability of the crystalline forms of p-toluenesulfonic acid salt in different water activity conditions was studied in slurry experiments where crystalline form 3 was stirred for 2-3 weeks at 5° C., 25° C. and 40° C. in mixtures of ethanol:water, acetonitrile (ACN):water, methanol: water and methanol alone. 0.5 ml of solvent was added to 25 mg of each sample. The resulting crystalline form was determined by XRPD. The results are shown in Table 17.

TABLE 17
Stability of the crystalline forms of p-toluenesulfonic
acid salt in different water activity conditions

| | | | | Resulting | Resulting | Resulting |
|---|---|---|---|---|---|---|
| Starting | | Solvent | Water | form | form | form |
| material | Solvent | ratio | activity | 5° C. | 25° C. | 40° C. |
| Form 3 | EtOH:water | 96.7:3.3 | 0.22 | Form 1 | Form 1 | Form 1 |
| | | 92.8:7.2 | 0.40 | Form 1 | Form 1 | Form 1 |
| | | 83:17 | 0.64 | Form 2 | Form 2 | Form 2 |
| | | 30:70 | >0.9 | Form 2 | Form 2 | Form 2 |
| | ACN:water | 96.3:3.7 | 0.58 | Form 2 | Form 2 | Form 2 |
| | | 92.1:7.9 | 0.81 | Form 2 | Form 2 | Form 2 |
| | MeOH:water | 95.3:4.7 | 0.16 | Form 1 | Form 1 | Form 1 |
| | | 90:10 | 0.30 | Form 1 | Form 1 | Form 1 |
| | | 77:23 | 0.52 | Form 2 | Form 2 | Form 1 |
| | | 36:64 | 0.83 | Form 2 | Form 2 | Form 2 |
| | MeOH | — | — | Form 1 | Form 1 | Form 1 |

The results show that crystalline form 1 of p-toluenesulfonic acid salt is favoured in conditions of low water activity (up to about 0.4). Crystalline form 2 of p-toluenesulfonic acid salt is favoured in conditions of higher water activity (higher than about 0.5) thus being suitable for pharmaceutical processing comprising wet (aqueous) granulation. Crystalline form 3 is unstable in these conditions.

### Example 29. Physical Stability of Salts at Storage Conditions

Short-term physical stability of various salts was studied by exposure at 40° C./75% RH for 2 weeks followed by measurement by XRPD. The results are shown in Table 18.

TABLE 18
Physical stability of salts after 40°
C./75% RH for 2 weeks as determined by XRPD

| Salt | Form after 2 weeks |
|---|---|
| p-Toluenesulfonic acid salt (form 1) | No change |
| p-Toluenesulfonic acid salt (form 2) | No change |
| 2-Naphthalenesulphonic acid salt (form 1) | No change |
| Hydrobromic acid salt (form 1) | No change |
| Hydrochloric acid salt (form 1) | No change |
| Benzenesulfonic acid salt (form 1) | No change |
| Methanesulfonic acid salt (form 1) | No change |
| Oxalic acid salt (form 1) | Form 1 + extra peaks |
| Oxalic acid salt (form 2) | No change |
| Oxalic acid salt (form 3) | Form 3 + extra peaks |
| Oxalic acid salt (form 4) | No change |
| Maleic acid salt (form 1) | Form 1 + extra peaks |

1. A salt of 5-((1-(methylsulfonyl)piperidin-4-yl)methoxy)-2-((5-(trifluoro-methyl)isoindolin-2-yl)methyl)-4H-pyran-4-one (I) with an acid selected from the group consisting of
p-toluenesulfonic acid,
2-naphthalenesulfonic acid,
hydrobromic acid,
hydrochloric acid,
methanesulfonic acid
benzenesulfonic acid,
oxalic acid,
phosphoric acid, and
maleic acid.

2. The salt according to claim 1, which is a salt of 5-((1-(methylsulfonyl)piperidin-4-yl)methoxy)-2-((5-(trifluoromethyl)isoindolin-2-yl)methyl)-4H-pyran-4-one (I) with an acid selected from the group consisting of
p-toluenesulfonic acid,
2-naphthalenesulfonic acid, and
hydrobromic acid.

3. The salt according to claim 1 which is crystalline.

4. The salt according to claim 3, which is a crystalline p-toluenesulfonic acid salt of 5-((1-(methylsulfonyl)piperidin-4-yl)methoxy)-2-((5-(tri-fluoromethyl)isoindolin-2-yl)methyl)-4H-pyran-4-one (I).

5. The salt according to claim 4, which is of crystalline form 1 of p-toluenesulfonic acid salt having an X-ray powder diffraction pattern characterized by peaks, expressed in degrees 2-theta (±0.2), at 4.4, 7.6, 11.5, 16.4, 17.7, 20.2 and 24.6.

6. (canceled)

7. The salt according to claim 5, wherein the crystalline form 1 of p-toluenesulfonic acid salt has the following unit cell parameters at T=293 (2) K:

| | | |
|---|---|---|
| Crystal system | Monoclinic | |
| Space group | P2₁/c | |
| Unit cell dimensions | a = 6.4221(18) Å | α = 90° |
| | b = 12.162(4) Å | β = 93.06(3)° |
| | c = 40.297(12) Å | γ = 90° |
| Volume | V = 3143.0(16) Å³ | |
| Z | 4 | |
| Goodness-of-fit | 1.188 | |
| R factor | 0.2435 | |
| Morphology | Thin plate | |

8. The salt according to claim 4, which is of crystalline form 2 of p-toluenesulfonic acid salt having an X-ray powder diffraction pattern characterized by peaks, expressed in degrees 2-theta (±0.2), at 4.4, 6.5, 13.0, 18.8, 20.1 and 22.4.

9. (canceled)

10. The salt according to claim 3, which is a crystalline 2-naphthalenesulfonic acid salt of 5-((1-(methylsulfonyl)piperidin-4-yl)methoxy)-2-((5-(tri-fluoromethyl)isoindolin-2-yl)methyl)-4H-pyran-4-one (I).

11. The salt according to claim 10, which is of crystalline form 1 of 2-naphthalenesulfonic acid salt having an X-ray powder diffraction pattern characterized by peaks, expressed in degrees 2-theta (±0.2), at 4.4, 11.1, 18.2, 18.6, 20.1 and 22.5.

12. (canceled)

13. The salt according to claim 3, which is a crystalline hydrobromic acid salt of 5-((1-(methylsulfonyl)piperidin-4-yl)methoxy)-2-((5-(trifluoromethyl)-isoindolin-2-yl)methyl)-4H-pyran-4-one (I).

14. The salt according to claim 13, which is of crystalline form 1 of hydrobromic acid salt having an X-ray powder diffraction pattern characterized by peaks, expressed in degrees 2-theta (±0.2), at 4.7, 7.0, 11.5, 18.5, 20.8 and 22.3.

15. (canceled)

16. A method of preparing a crystalline salt according to claim 4, comprising dissolving 5-((1-(methylsulfonyl)piperidin-4-yl)methoxy)-2-((5-(trifluoromethyl)isoindolin-2-yl)methyl)-4H-pyran-4-one (I) and p-toluenesulfonic acid in acetonitrile, 1-propanol, 2-butanol or ethanol, cooling the mixture and isolating the crystalline product.

17. A method of preparing a crystalline salt according to claim 8, comprising dissolving 5-((1-(methylsulfonyl)piperidin-4-yl)methoxy)-2-((5-(trifluoromethyl)-isoindolin-2-yl)methyl)-4H-pyran-4-one (I) and p-toluenesulfonic acid in a mixture of acetonitrile and water, cooling the mixture and isolating the crystalline product.

18. The method according to claim 17, wherein the amount of water is from about 5% to about 15%, per volume of the acetonitrile/water mixture.

19. (canceled)

20. The method according to claim 17, wherein the amount of 5-((1-(methylsulfonyl)piperidin-4-yl)methoxy)-2-((5-(tri-fluoromethyl)isoindolin-2-yl)methyl)-4H-pyran-4-one (I) is from about 5 g to about 15 g, per 100 ml of the acetonitrile/water mixture.

21. (canceled)

22. The method according to claim 17, wherein p-toluenesulfonic acid is used in about equivalent molar amount in relation to the amount of 5-((1-(methylsulfonyl)piperidin-4-yl)methoxy)-2-((5-(trifluoromethyl)isoindolin-2-yl)methyl)-4H-pyran-4-one (I).

23. The method according to claim 17, wherein the crystalline product is washed with water and dried at about 20-60° C.

24. (canceled)

25. A method of preparing a crystalline salt according to claim 10, comprising dissolving 5-((1-(methylsulfonyl)piperidin-4-yl)methoxy)-2-((5-(trifluoromethyl)isoindolin-2-yl)methyl)-4H-pyran-4-one (I) and 2-naphthalenesulfonic acid in acetonitrile, cooling the mixture and isolating the crystalline product.

26. A method of preparing a crystalline salt according to claim 13, comprising dissolving 5-((1-(methylsulfonyl)piperidin-4-yl)methoxy)-2-((5-(trifluoromethyl)isoindolin-2-yl)methyl)-4HI-pyran-4-one (I) and hydrobromic acid in acetonitrile, cooling the mixture and isolating the crystalline product.

27. A pharmaceutical composition comprising a salt according to claim 1 as an active ingredient together with one or more excipients.

28. The pharmaceutical composition according to claim 27, which is in the form of a tablet, capsule, granule, powder or suspension.

29. (canceled)

30. (canceled)

31. The pharmaceutical composition according to claim 28, which is in form of a tablet or capsule prepared by wet granulation.

32. A method for treatment of a hormonally regulated cancer comprising administering to a subject in need thereof a therapeutically effective amount of a salt according to claim 1.

33. The method according to claim 32, wherein the hormonally regulated cancer is selected from the group consisting of prostate cancer and breast cancer.
